(19) United States
(12) Patent Application Publication
(10) Pub. No.: US 2007/0185077 A1
(43) Pub. Date: Aug. 9, 2007

(54) Methods of treating, preventing and inhibiting cancer metastasis and tumor formation

(75) Inventors: Katherine Weilbaecher, University City, MO (US); Suzanne Bakewell, Philadelphia, PA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University

(21) Appl. No.: 10/564,945

(22) PCT Filed: Jul. 15, 2004
(86) PCT No.: PCT/US04/22697

§ 371 Date: Jan. 9, 2007

Related U.S. Application Data
(60) Provisional application No. 60/487,325, filed on Jul. 15, 2003.

(51) Int. Cl. A61K 31/55; A61K 31/4747; A61K 31/407; A61K 31/397

(52) U.S. Cl. 514/210.16; 514/212.02; 514/278; 514/409

(57) ABSTRACT

Disclosed are methods for treating, preventing or inhibiting tumor cell metastasis, tumor cell formation, and destroying tumors in a subject comprising administering to the subject in need of such therapy a therapeutically effective amount of an activated aIIbβ3 receptor antagonist and by transplanting affected bone marrow with β3−/−marrow or controlling the expression of β3 integrin in vitro or in vivo. The activated allbβ3 receptor antagonist is preferably a spiro compound.

### CROSS-REFERENCES TO RELATED APPLICATIONS

This application claims priority from Provisional Application Ser. No.60/487,325 filed on Jul. 15, 2003, which is incorporated herein by reference in its entirety.

### STATEMENT REGARDING FEDERALLY SPONSORED RESEARCH OR DEVELOPMENT

This invention was made in part with Government support under National Institutes of Health Grant K08ag00853. The Government has certain rights in the invention.

### INCORPORATION-BY-REFERENCE OF MATERIAL SUBMITTED ON A COMPACT DISC

Not Applicable.

### BACKGROUND OF THE INVENTION

1. Field of the Invention

The present invention relates generally to inhibition, prevention and therapy of tumor cell metastasis, tumor growth and tumor destruction, and in particular, provides methods for inhibiting, preventing and treating tumor cell metastasis to bone by specifically antagonizing activated platelet α_{IIb}β₃ integrin receptors and by selectively removing β₃ integrin from a subject by transplantation or other methods.

2. Description of the Related Art

Metastasis to and invasion of cortical bone is a cause of significant morbidity and mortality in patients with solid tumors. While the mechanisms underlying these processes are currently unknown, there is growing evidence that integrins play a crucial role in metastasis and tumor formation. Integrins are a family of αβ heterodimers that mediate adhesion of cells to extracellular matrix proteins and to other cells. The integrin family consists of 15 related known α subunits (β₁, α₂, α₃, α₄, α₅, α₆, α₇, α₈, α₉, α_{E}, α_{V}, α_{IIb}, α_{L}, α_{M}, and α_{X}) and 8 related known β subunits (β₁, β₂, β₃, β₄, β₅, β₆, β₇, and β₈). Integrin α and β subunits are known to exist in a variety of pairings. Integrin ligand specificity is determined by the specific pairing of the α and β subunits, although some redundancy exists as several of the integrins are known to bind the same ligand.

Many biological responses are dependent at least to some extent upon integrin-mediated adhesion and cell migration, Including embryonic development, hemostasis, clot retraction, mitosis, angiogenesis, cell migration, inflammation, immune response, leukocyte homing and activation, phagocytosis, bone resorption, tumor growth and metastasis, atherosclerosis, restenosis, wound healing, viral infectivity, amyloid toxicity, programmed cell death and the response of cells to mechanical stress.

The β₃ integrin subunit has been implicated in the development of metastases because of its critical role in osteoclastic bone resorption, its role in platelet aggregation and tumor/platelet interactions, and its role in tumor associated angiogenesis. β₃ integrin (also known as human endothelial glycoprotein, GP3A, GPIIIa, ITGB3, CD61 and platelet glycoprotein 3a) is the common beta subunit partner of the members of the β₃ subfamily of integrins. This subfamily consists of two members, the vitronectin receptor, and the fibrinogen receptor and cells expressing this class of integrin receptor can adhere to various matrix proteins and participate in cytoadhesion driven cellular responses.

The activation of the vitronectin receptor has been shown to promote cellular migration and to provide signals in the regulation of cell proliferation and differentiation and to potentiate the effects of insulin. Upregulation of the vitronectin receptor is associated with pathological conditions such as vascular restenosis, excessive bone resorption, and the process of angiogenesis during malignant melanomas.

β₃ integrin, in conjunction with integrin alpha IIb, also forms the fibrinogen receptor (α_{IIb}/β₃) which mediates platelet aggregation. This receptor is basally inactive but can be activated by several agonists causing it to bind fibrinogen which then forms cross-bridges to fibrinogen receptors on adjacent cells. This receptor has also been shown to bind other proteins including fibronectin, von Willebrand factor (vWf) and vitronectin.

α₃ integrin, in conjunction with integrin alpha v, forms the vitronectin receptor (α_{V}β₃). This heterodimeric receptor is localized to platelets, endothelial cells, monocytes, macrophages and osteoclasts with the highest expression found in the osteoclasts. The vitronectin receptor functions to mediate the adhesion of cells to vitronectin, and a variety of extracellular matrix proteins. It is through a specific tripeptide sequence referred to as the RGD sequence, so named because of its amino acid composition (arginine-glycine-aspartic acid), that receptor-protein binding occurs.

Therapeutic agents in the art which are designed to affect the function of receptors containing the β₃ integrin subunit interfere with the binding properties of the receptor. As such, several antagonists have been reported in the art which include synthetic compounds and their derivatives, antibodies, and peptidomimetics, all of which antagonize receptor-ligand binding.

One category of such antagonists that targets the vitronectin receptor are peptidomimetics designed to block the interactions between the receptor and RGD-containing proteins. One such antagonist is Abciximab (ReoPro®, Eli Lilly and Co., Indianapolis, Ind.), the Fab fragment of the chimeric human-murine monoclonal antibody 7E3. Abciximab binds to the α_{IIb}β₃ receptor of human platelets and inhibits platelet aggregation. The mechanism of action is thought to involve steric hindrance and/or conformational effects to block access of large molecules to the receptor rather than direct interaction with the RGD binding site of IIb/IIIa. Abciximab also binds with similar affinity to the vitronectin (α_{V}β₃) receptor found on platelets and vessel wall endothelial and smooth muscle cells. Thus, Abciximab is not specific to the α_{IIb}β₃ receptor which could produce unwanted side effects. Although Abciximab is indicated as an adjunct to percutaneous coronary intervention for the prevention of cardiac ischemic complications, a potentially dangerous human antibody response to the chimeric antibody occurred in approximately 6% of patients (Clemetson and Clemetson, Cell. Mol. Life Sci. (1998) 54:502-513).

Monoclonal antibodies to both β₃ integrin-containing receptors have also been reported in the literature. Recently it was demonstrated that prostate carcinoma cells express the α_{IIb}β₃ receptor and that antibodies to this receptor were capable of inhibiting the invasive properties of the carcinoma cells (Trikha et al., Cancer Res. (1996) 56:5071-5078). However, such antibodies were not disclosed to differentially bind to activated versus unactivated states of the α_{IIb}β₃ receptors. Thus, the lack of specificity in binding could produce inaccurate data analysis when studying the antibodies in vitro and unwanted side effects when administerd to a patient in vivo.

In addition, monoclonal antibodies produced by three different hybridoma cell lines that target the αᵥβ₃ (vitronectin) receptor and inhibit the binding of fibronectin or vitronectin to osteoclasts are also disclosed in U.S. Pat. Nos. 5,578,704, 5,652,109 and 5,652,110. These antibodies are used to detect the αᵥβ₃ integrin and to treat disease conditions characterized by excessive bone resorption.

Antisense technology has been demonstrated as an effective means for reducing the expression of specific gene products can be used in a number of applications for the modulation of β₃ integrin expression as disclosed in U.S. Pat. No. 6,037,176.

αᵥβ₃ integrin antagonists have also been disclosed in U.S. Pat. No. 6,521,593 which are capable of inhibiting brain tumor metastasis. The antagonists described in the patent are antibodies and peptides which disrupt the RGD binding site by direct or competitive binding to a RGD ligand.

Florida Hospital Cancer Institute discloses in their Internet literature glycoprotein complex IIb/IIIa inhibition of angiogenesis, Le. blood vessel growth and blood vessel permeability, with a tumor/platelet adhesion inhibitor XV454. It is taught that tumor-platelet adhesion upregulates VEGFR expression, thus enhancing angiogenesis and metastasis. However, XV454 is a nonspecific inhibitor of two classes of GPIIb/IIIa receptors. Class I compounds (e.g., roxifiban, DMP802, and XV454) bind to both resting and activated platelet GPIIb/IIIa receptors with comparable K_{d} values and relatively slow platelet dissociation rates. Class II compounds, on the other hand, (e.g., L734217, MK852, and DMP728) bind with much higher affinity to the activated form of GPlb/IIIa than to the resting form and have relatively fast platelet dissociation rates (e.g., for selective antagonists such as L734217, K_{d}(activated)=5 nM and K_{d}(resting)=620 nM). Thus, the lack of specificity in antagonizing both the activated an unactivated receptor could produce inaccurate data analysis when studying the antagonists in vitro and unwanted side effects when administered to a patient in vivo. Such compositions also affect platelet binding to tumor cells as opposed to platelet/platelet binding.

Overall, the above references focus on the function or action of metastatic tumor cells alone, but do not teach treating, preventing or inhibiting metastasis by protecting the organs affected by tumor cell metastasis or tumor cell invasion. Such a protective mechanism avoids many of the problems associated with treating, preventing or inhibiting metastasis by focusing on complex and not-well-understood tumor cell function at the stages of tumor cell genesis, mobility, infiltration and tumor cell growth among unprotected cells. Therefore, what is needed are methods of treating, preventing or inhibiting metastasis and tumor cell growth by protecting organs affected or likely to be affected by tumor cell metastasis or invasion.

### BRIEF SUMMARY OF THE INVENTION

Accordingly, it is an object of the invention to overcome these and other problems associated with the related art. These and other objects, features and technical advantages are achieved by administering an activated αIIbflβ₃ receptor antagonist to a patient affected by tumor cell metastasis. Surprisingly, such compounds specifically protect organs, including the skeletal system and more specifically bone cells, from tumor cell metastasis and tumor cell invasion. In particular, the spiro compounds of the present invention were found to protect healthy organ systems or parts of healthy organ systems in contrast to prior art compounds which target tumors and tumor cells themselves. Such spiro compounds were also found to reverse tumor growth in vivo.

Therefore, the invention provides a method for treating, preventing or inhibiting tumor cell metastasis in a subject comprising administering to the subject in need of such therapy a therapeutically effective amount of an activated α_{IIb}β₃ receptor antagonist. In accordance with one aspect of the invention, the tumor cell metastasis targets an organ system, skeletal system, bone, or bone cell of the subject. In another aspect, the antagonist is a platelet-specific activated α_{IIb}β₃ receptor antagonist. Preferably, the platelet-specific activated α_{IIb}β₃ receptor antagonist is a spiro compound. More preferably, the spiro compound is represented by the formula:
Q-(L)_{Z}-Z-R₃
wherein

Z is a spirocyclic nucleus selected from the group consisting of Nucleus (A), (B), (C), or (D) represented by the formulas: 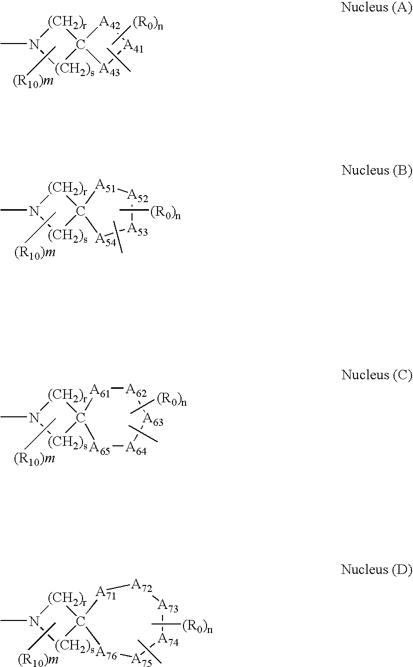
wherein

the group Q-(L)_{z}- is bound to the nitrogen containing ring of nuclei (A), (B), (C), or (D) and the group R₃ is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆; or

the group R₃ is bound to the nitrogen containing ring and the group Q-(L)_{z}- is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆;

r and s are independently a number from zero to 5 with the proviso that not both r or s are 0 and (r+s) is not more than 6, and z is zero or one;

atoms A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆ are independently selected from carbon, nitrogen, oxygen or sulfur, provided that at least one of said atoms is carbon;

provided that the hydrogens of the nitrogen containing part of the spirocycle Z may be substituted by a number of m substituents R₁₀, wherein;

m is a number from zero to (r+s); and

R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₁₀ may be ═══O or ═══S;

n is a number from zero to 3 in Z of having nuclei (A), or a number from zero to 4 in Z having nuclei (B), a number from zero to 5 in Z having nuclei (C), or a number from zero to 6 in Z having nuclei (D);

R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two Ro may be ═══O or ═══S; and

-(L)- is a bond or a divalent substituted or unsubstituted chain of from 1 to 10 atoms selected from the group consisting of carbon, nitrogen, sulfur, and oxygen;

Q Is a basic group containing one or more basic radicals; and

R₃ is an acidic group containing one or more acid radicals;

or a pharmaceutically acceptable salt, solvate or pro-drug thereof.

The spiro compound may also be represented by the formula: 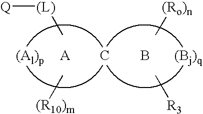
wherein

atoms Aᵢ and Bⱼ are independently selected from carbon, nitrogen, oxygen or sulfur, provided that at least one atom of Aᵢ is carbon, and at least one atom Bⱼ is carbon;

optionally, the rings of the spirobicycle formed by Aᵢ and Bⱼ , respectively, are partly unsaturated;

p and q are independently numbers from 2 to 6;

m is a number from zero to p;

R₁₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₁₀ may be ═══O or ═══S, if p is 2 or one or two R₁₀ may be ═══O or ═══S, if p is a number from 3 to 6;

n is the number from zero to q;

R₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₀ may be ═══O or ═══S, if q is 2 or one or two R₀ may be ═══O or ═══S, if q is a number from 3 to 6;

-(L)- is a bond or a divalent substituted or unsubstituted chain of from 1 to 10 atoms selected from the group consisting of carbon, nitrogen, sulfur, and oxygen;

Q is a basic group containing one or more basic radicals; and

R₃ is an acidic group containing one or more acid radicals;

or a pharmaceutically-acceptable salt, solvate or pro-drug thereof.

In addition, the spiro compound may be represented by the formula: 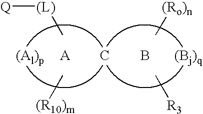
wherein

the spirocycle having (Aᵢ)ₚ, C, and (Bⱼ )_{q} is

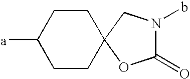

m is a number from zero to 9;

R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;

n is a number from zero to 2;

R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;

wherein Q-(L) is attached at a, and R₃ is attached at b;

-(L)- is a bond or a substituted or unsubstituted chain selected from the group consisting of CO, CO(C₁-C₆ alkyl), O(C₁-C₆ alkyl), NHCO, and C₁-C₆ alkyl;

Q is a basic group selected from the group consisting of amino, imino, amidino, hydroxyamidino, N-alkylamidine, N,N′-dialkylamidine, N-arylamidine, aminomethyleneamino, aminomethylamino, guanidino, aminoguanidino, alkylamino, dialkylamino, trialkylamino, alkylideneamino, pyrrolyl, imidazolyl, pyrazolyl, pyridyl, pyrazinyl, pyrimidinyl, indolizinyl, isoindolyl, 3H-indolyl, indolyl, 1H-indazolyl, purinyl, 4H-quinolizinyl, isoquinolyl, quinolyl, phthalazinyl, naphthyridinyl, quinoxalinyl, quinazolinyl, cinnolinyl, amide, thioamide, benzamidino, pteridinyl, 4aH-carbozolyl, carbozolyl, beta-carbolinyl, phenanthridinyl, acridinyl, phenanthrolinyl, phenazinyl, phenarsazinyl, phenothiazinyl, pyrrolinyl, imidazolidinyl, imidazolinyl, pyrazolidinyl, pyrazolinyl, piperidyl, piperazinyl, indolinyl, isoindolinyl quinuclidinyl, morpholinyl, any of the foregoing radicals substituted on a benzene ring, optionally substituted with R_{2c}, wherein R_{2c} is hydrogen or halogen and any of the foregoing radicals substituted by amino, imino, amidino, hydroxyamidino, aminomethyleneamino, iminomethylamino, guanidino, alkylamino, dialkylamino, trialkylamino, tetrahydroisoquinoline, dihydrosioindole, alkylideneamino or

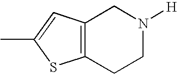

R₃ is an acidic group selected from the group consisting of C₂ R₅, (C₁-C₆ alkyl)CO₂ R₅, CO(C₁-C₆ alkyl)CO₂ R₅, CONH(C₁-C₆ alkyl)CO₂ R₅, (C₁-C₆ alkyl)CH(NHR₄)CO₂ R₅, CO(C₁-C₆ alkyl)CH(NHR₄)CO₂ R₅, or CONH(C₁-C₆ alkyl)CH(NHR₄)CO₂ R₅, wherein R₄ is SO₂ (C₁-C₆ alkyl), SO₂ aryl, or SO₂ (substituted aryl); and

R₅ is hydrogen, C₁-C₆ alkyl, aryl, or substituted aryl;

or a pharmaceutically acceptable salt, solvate or pro-drug thereof.

In accordance with another aspect of the invention, the spiro compound may be represented by the formula: 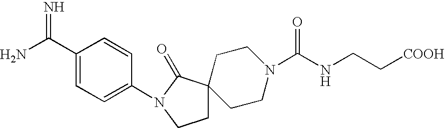
or a pro-drug thereof.

In accordance with yet another aspect of the present invention, a pro-drug may be represented by the formula:

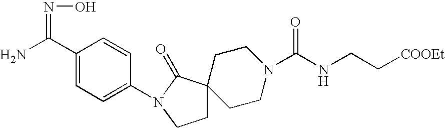

In accordance with a further aspect of the invention, a method is provided for preventing or inhibiting tumor cell formation in a subject comprising administering to the subject in need of such therapy a therapeutically effective amount of an activated α_{IIb}β₃ receptor antagonist. In yet another aspect of the invention, a method is provided for destroying a tumor in a subject comprising administering to the subject in need of such therapy a therapeutically effective amount of an activated α_{IIb}β₃ receptor antagonist.

In another aspect of the invention, a method is provided for treating, preventing or inhibiting tumor cell metastasis to bone in a subject comprising replacing substantially all bone marrow affected by tumor cell metastasis transplant in the subject, wherein said bone marrow is replaced with β₃^{-/-} bone marrow.

In yet another aspect of the invention, a method is provided for treating, preventing or reversing tumor metastasis or formation comprising modulating β₃ integrin expression. Preferably, the modulating β₃ integrin expression comprises decreasing the β₃ integrin expression in a mammalian cell. In one aspect, decreasing the expression comprises transforming the cell to express a polynucleotide anti-sense to at least a portion of an endogenous polynucleotide encoding β₃ integrin. In another aspect, decreasing the expression comprises transfecting the cell with a polynucleotide anti-sense to at least a portion of an endogenous polynucleotide encoding 62 ₃ integrin. Preferably, decreasing the expression comprises transfecting a cell with a siRNA targeting at least a portion of an endogenous polynucleotide encoding β₃ integrin.

These and other features, aspects and advantages of the present invention will become better understood with reference to the following description, examples and appended claims.

### BRIEF DESCRIPTION OF THE SEVERAL VIEWS OF THE DRAWINGS

FIG. 1. This figure depicts results of experiments using β₃^{-/-} mice and protection from osteolytic bone metastases. FIG. 1(a) shows bone metastases in β₃^{+/+} but not in β₃^{-/-} mice. FIG. 1(b) shows a B16 LV injected β^{+/+} mouse where arrows mark TRAP positive osteoclasts recruited to B16 tumor cells. FIG. 1(c) shows a comparison of mice with bone metastases of B16 cells for β₃^{+/+} compared to β₃^{-/-}mice. FIG. 1(d) shows a comparison of mice with pigmented visceral metastases of B16 melanoma cells in β₃^{+/+} compared to β₃^{-/-} mice. FIG. 1(e) shows B16 osteolytic bone invasion in β₃^{+/+} mice.

FIG. 2. This figure depicts results of experiements using β₃^{-/-} mice and protection from osteolytic bone invasion after direct inoculation of tumor cells into the bone marrow cavity. FIG. 2(a) shows TRAP/Hematoxalin staining of tibial cross section 14 days after B16 or saline IT injection. FIG. 2(b) shows histomorphometric analysis of trabecular bone area for saline (S) and B16(T) IT injected tibia. FIG. 2(c) shows trabecular bone area 14 days after B16 intra-tibial injection.

FIG. 3. This figure depicts bone marrow transplantation of β₃^{-/-} marrow conferring protection from osteolytic metastases. FIG. 3(a) shows TRAP staining of femur 10 days after irradiation in untransplanted control mouse. FIG. 3(b) shows in vitro TRAP staining of cultured osteoclasts. FIG. 3(c) shows bleeding times performed on β₃^{-/-} mice 3 weeks after transplantation with β₃^{+/+} marrow. FIG. 3(d) shows percentage of transplanted mice with visible bone metastases (B) and visceral metastases (V) 14 days after LV injection of B16 cells. FIG. 3(e) shows transplanted knockout mice having reduced metastases. FIG. 3(f) shows bone marrow transplantation restoring bone metastases in β₃^{-/-} mice.

FIG. 4. This figure depicts osteoclast defective src^{-/-} mutant mice developing bone lesions without tumor associated bone destruction. FIG. 4(a) shows visible pigmented B16 melanoma cells bone lesions were seen in src^{+/+} and src^{-/-} mice. FIG. 4(b) shows histology of TRAP-stained tibias from saline LV injected src^{+/+} and src^{-/-} mice compared to B16 LV injected mice. FIG. 4(c) shows histomorphometry results in tumor-induced trabecular bone loss in src^{+/+} mice.

FIG. 5. This figure depicts an αIIbβ₃ inhibitor of platelet aggregation reducing metastases in β₃^{+/+} Mice. FIG. 5(a) shows B16 cells spreading on fibrinogen coated surface (diamond) were not inhibited by ML728 (square) but completely inhibited by RGD peptide (triangle). FIG. 5(b) shows ML464 administration to β₃^{+/+} mice results in a decrease of metastases in inhibitor treated mice compared to placebo treated mice. FIG. 5(c) shows B16 cells added to unactivated platelets induce platelet aggregation where the arrow represents the addition of B16 cells to platelets, the blue line represents microaggregates, and black line is total aggregates (micro and large). FIG. 5(d) shows calcein labeled fluorescent mouse platelets adhere to unlabeled B16 tumor cells (arrows). The left panel shows aggregates of platelets and tumor cells and the right panel shows inhibition of tumor cell and platelet aggregation/clumping but not platelet-tumor cell adhesion after adding ML728. FIG. 5(e) shows B16 cells adhering to platelets in the presence (+Fib) or absence (−Fib) of fibrinogen, which was not inhibited by ML728 (Inh).

### DETAILED DESCRIPTION OF THE INVENTION

Abbreviations and Definitions

To facilitate understanding of the invention, a number of terms and abbreviations as used herein are defined below have the following meanings:

Controlled-Release Component: As used herein, the term “controlled-release component” refers to a compound or compounds, including, but not limited to, polymers, polymer matrices, gels, permeable membranes, liposomes, microspheres, or the like, or a combination thereof, that facilitates the controlled-release of an active ingredient.

Pharmaceutically Acceptable: As used herein, the term “pharmaceutically acceptable” means approved by a regulatory agency of the Federal or a state government or listed in the U.S. Pharmacopeia or other generally recognized pharmacopeia for use in animals, and more particularly in humans.

Pharmaceutically Acceptable Carrier: As used herein, the term “carrier” refers to a diluent, adjuvant, excipient, or vehicle with which a composition is administered. Such carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when a pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier.

Pharmaceutically Acceptable Salt: As used herein, the term “pharmaceutically acceptable salt” includes those salts of a pharmaceutically acceptable composition formed with free amino groups such as those derived from hydrochloric, phosphoric, acetic, oxalic, tartaric acids, and those formed with free carboxyl groups such as those derived from sodium, potassium, ammonium, calcium, ferric hydroxides, isopropylamine, triethylamine, 2-ethylamino ethanol, histidine, and procaine. If the composition is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. If the composition is acidic, salts may be prepared from pharmaceuticaily acceptable organic bases including, but not limited to, lysine, N,N′-dibenzylethylenediamine, chloroprocaine, choline, diethanolamine, ethylenediamine, meglumine (N-methylglucamine) and procaine. Suitable inorganic bases include, but are not limited to, alkaline and earth-alkaline metals such as aluminum, calcium, lithium, magnesium, potassium, sodium and zinc.

Pro-drug: As used herein, the term “pro-drug” refers to any compound which releases an active drug in vivo when such a compound is administered to a mammalian subject. Pro-drugs can be prepared, for example, by functional group modification of a parent drug. The functional group may be cleaved in vivo to release the active parent drug compound. Pro-drugs include, for example, compounds in which a group that may be cleaved in vivo is attached to a hydroxy, amino or carboxyl group in the active drug. Examples of pro-drugs include, but are not limited to esters (e.g., acetate, methyl, ethyl, formate, and benzoate derivatives), carbamates, amides and ethers. Methods for synthesizing such pro-drugs are known to those of skill in the art.

Spiro compound: As used herein, the term “spiro compound” refers to those compositions and compounds disclosed by U.S. Pat. Nos. 6,291,469 and 6,528,534, incorporated herein by reference in their entirety, and related patents and patent applications. Particularly preferred spiro compounds have a Spiro nucleus formed from two fused rings, A and B, represented by the following formula: 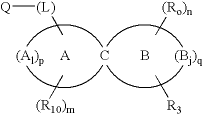
Pharmaceutically acceptable salts, solvates and pro-drug derivatives thereof are also covered by this definition. Substituents, analogs, precursors and reactants are disclosed in U.S. Pat. No. 6,528,534, and related patents and patent applications, and are encompassed within the scope of the present invention. Examples of such substituents, analogs, precursors and reactants may be found in the definitions of “alkyl”, “halosubstituted alkyl”, “aryl”, “substituted aryl”, “arylalkyl”, “alkenyl”, “alkylene”, “alkenylene”, “alkynylene”, “amidino”, “basic radical”, “basic group”, “acidic group”, and “non-interfering substituent” of this patent. Renderings of representative spiro nuclei, substituted spirocyclic nuclei and individual substituents are hereby incorporated by reference in their entirety.

Also included in the definition of spiro compound, although less preferred, are those compositions identified as such in U.S. Pat. Nos. 6,552,079, 6,548,517, 6,399,627, 6,245,809, 5,968,902, 5,958,732, 5,935,926, 5,843,897, 5,807,825, 5,786,333, 5,770,564, 5,759,999, 5,756,451, 5,736,339, 5,686,571, 5,686,570, 5,686,569, 5,686,568, 5,686,567, 5,686,566, 5,496,724, 5,344,783, and 5,318,899.

Therapeutically Effective Amount: As used ,herein, the term “therapeutically effective amount” refers to those amounts that, when administered to a particular subject in view of the nature and severity of that subject's disease or condition, will have the desired therapeutic effect, e.g., an amount which will cure, or at least partially arrest or prevent the disease or condition. More specific embodiments are included in the Pharmaceutical Preparations and Methods of Administration section below.

Activated Platelet-Specific β₃ Integrin Inhibitors in Metastasis and Tumor Formation

The invention provides a method for treating, preventing or inhibiting tumor cell metastasis in a subject comprising administering to the subject in need of such therapy a therapeutically effective amount of an activated α_{IIb}β₃ receptor antagonist. The activated α_{IIb}β₃ receptor antagonists of the present invention protect healthy skeletal systems or parts of healthy skeletal systems from tumor metastasis and tumor growth. Such compounds were previously known to treat certain conditions affected by platelet binding such as the pathological effects of atherosclerosis, arteriosclerosis, acute myocardial infarction, chronic stable angina, unstable angina, transient ischemic attacks and strokes, peripheral vascular disease, arterial thrombosis, preeclampsia, embolism, restenosis following angioplasty, carotid endarterectomy, and anastomosis of vascular grafts. Thus, it is surprising that such compositions provide a protective effect from tumor cell metastasis and tumor growth.

Such activated α_{IIb}β₃ receptor antagonists protect organ systems, such as the skeletal system, from the growth of tumor metastases and from further metastasis. Unlike prior art methods which target the function or action of tumor cells alone, the present invention provides methods for treating, preventing or inhibiting metastasis by protecting the organs affected by tumor cell metastasis or tumor cell invasion. Specifically, such compounds protect the skeletal system, including bone and bone cells, from tumor cell metastasis and tumor cell invasion. Thus, one aspect of the invention provides a method for treating, preventing or inhibiting tumor cell metastasis targeting an organ system, skeletal system, bone, or bone cell of the subject.

As shown in the Examples below, the activated α_{IIb}β₃ receptor antagonist ML464 does not disrupt tumor-platelet adhesions, but prevents the development of larger tumor/platelet clumps in vitro. Without being limited to a particular theory, it is thought that such antagonists act by preventing plateletplatelet interaction and thus propagation of the clumps. Thus, the present invention also provides a method for treating, preventing or inhibiting tumor cell metastasis, tumor cell formation, and destroying tumors in a subject by administering a platelet-specific activated α_{IIb}β₃ receptor antagonist to a subject. As described below, the bioavailability, efficacy, safety and dosage and of individual activated α_{IIb}β₃ receptor antagonists, including spiro compounds, can be determined by those of skill in the art for administration to mammals, including humans.

Preferably, the platelet-specific activated α_{IIb}β₃ receptor antagonist is a spiro compound. More preferably, the spiro compound is represented by the formula:
Q-(L)_{Z}-Z-R₃
wherein

Z is a spirocyclic nucleus selected from the group consisting of Nucleus (A), (B), (C), or (D) represented by the formulas: 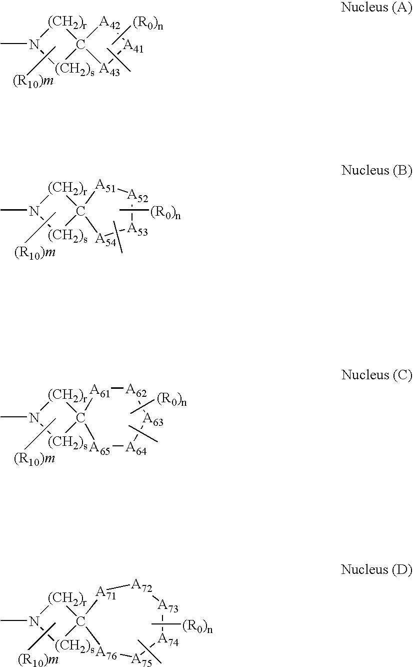
wherein

the group Q-(L)_{Z}- is bound to the nitrogen containing ring of nuclei (A), (B), (C), or (D) and the group R₃ is bound to the ring forrned by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆; or

the group β₃ is bound to the nitrogen containing ring and the group Q-(L)_{Z}- is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆;

r and s are independently a number from zero to 5 with the proviso that not both r or s are 0 and (r+s) is not more than 6, and z is zero or one;

atoms A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₄, or A₇₆ are independently selected from carbon, nitrogen, oxygen or sulfur, provided that at least one of said atoms is carbon;

provided that the hydrogens of the nitrogen containing part of the spirocycle Z may be substituted by a number of m substituents R₁₀, wherein;

m is a number from zero to (r+s); and

R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₁₀ may be ═══O or ═══S;

n is a number from zero to 3 in Z of having nuclei (A), or a number from zero to 4 in Z having nuclei (B), a number from zero to 5 in Z having nuclei (C), or a number from zero to 6 in Z having nuclei (D);

R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₀ may be ═══O or ═══S; and

-(L)- is a bond or a divalent substituted or unsubstituted chain of from 1 to 10 atoms selected from the group consisting of carbon, nitrogen, sulfur, and oxygen;

Q is a basic group containing one or more basic radicals; and

β₃ is an acidic group containing one or more acid radicals;

or a pharmaceutically acceptable salt, solvate or pro-drug thereof.

The spiro compound may also be represented by the formula: 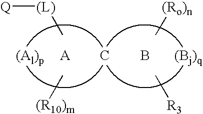
wherein

atoms Aᵢ and Bⱼ are independently selected from carbon, nitrogen, oxygen or sulfur, provided that at least one atom of Aᵢ is carbon, and at least one atom Bⱼ is carbon;

optionally, the rings of the spirobicycle formed by Aᵢ and Bⱼ , respectively, are partly unsaturated;

p and q are independently numbers from 2 to 6;

m is a number from zero to p;

R₁₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one Rl₀ may be ═══O or ═══S, if p is 2 or one or two R₁₀ may be ═══O or ═══S, if p is a number from 3 to 6;

n is the number from zero to q;

R₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₀ may be ═══O or ═══S, if q is 2 or one or two R₀ may be ═══O or ═══S, if q is a number from 3 to 6;

-(L)- is a bond or a divalent substituted or unsubstituted chain of from 1 to 10 atoms selected from the group consisting of carbon, nitrogen, sulfur, and oxygen;

Q is a basic group containing one or more basic radicals; and

R₃ is an acidic group containing one or more acid radicals;

or a pharmaceutically-acceptable salt, solvate or pro-drug thereof.

In addition, the spiro compound may be represented by the formula: 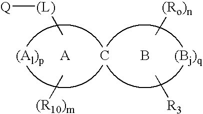
wherein

the spirocycle having (Aᵢ)ₚ, C, and (Bⱼ )_{q} is

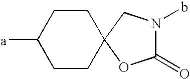

m is a number from zero to 9;

R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;

n is a number from zero to 2;

R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;

wherein Q-(L) is attached at a, and β₃ is attached at b;

-(L)- is a bond or a substituted or unsubstituted chain selected from the group consisting of CO, CO(C₁-C₆ alkyl), O(C₁-C₆ alkyl), NHCO, and C₁-C₆ alkyl;

Q is a basic group selected from the group consisting of amino, imino, amidino, hydroxyamidino, N-alkylamidine, N,N′-dialkylamidine, N-arylamidine, aminomethyleneamino, aminomethylamino, guanidino, aminoguanidino, alkylamino, dialkylamino, trialkylamino, alkylideneamino, pyrrolyl, imidazolyl, pyrazolyl, pyridyl, pyrazinyl, pyrimidinyl, indolizinyl, isoindolyl, 3H-indolyl, indolyl, 1H-indazolyl, purinyl, 4H-quinolizinyl, isoquinolyl, quinolyl, phthalazinyl, naphthyridinyl, quinoxalinyl, quinazolinyl, cinnolinyl, amide, thioamide, benzamidino, pteridinyl, 4aH-carbozolyl, carbozolyl, beta-carbolinyl, phenanthridinyl, acridinyl, phenanthrolinyl, phenazinyl, phenarsazinyl, phenothiazinyl, pyrrolinyl, imidazolidinyl, imidazolinyl, pyrazolidinyl, pyrazolinyl, piperidyl, piperazinyl, indolinyl, isoindolinyl quinuclidinyl, morpholinyl, any of the foregoing radicals substituted on a benzene ring, optionally substituted with R_{2c}, wherein R_{2c} is hydrogen or halogen and any of the foregoing radicals substituted by amino, imino, amidino, hydroxyamidino, aminomethyleneamino, iminomethylamino, guanidino, alkylamino, dialkylamino, trialkylamino, tetrahydroisoquinoline, dihydrosioindole, alkylideneamino or 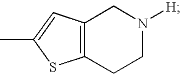
and

R₃ is an acidic group selected from the group consisting of CO₂R₅, (C₁-C₆ alkyl)CO₂ R₅, CO(C₁-C₆ alkyl)CO₂ R₅, CONH(C₁-C₆ alkyl)CO₂ R₅, (C₁-C₆ alkyl)CH(NHR₄)CO₂ R₅, CO(C₁-C₆ alkyl)CH(NHR₄)CO₂ R₅, or CONH(C₁-C₆ alkyl)CH(NHR₄)CO₂ R₅, wherein R₄ is SO₂ (C₁-C₆ alkyl), SO₂ aryl, or SO₂ (substituted aryl); and

R₅ is hydrogen, C₁-C₆ alkyl, aryl, or substituted aryl;

or a pharmaceutically acceptable salt, solvate or pro-drug thereof.

In particular, the spiro compound may be represented by the formula: 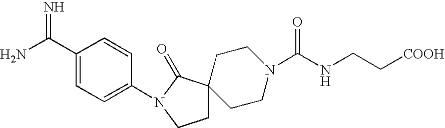
or a pro-drug thereof. The pro-drug may be represented by the formula where the COOEt moiety is converted to the active drug having a COOH moiety under predetermined conditions:

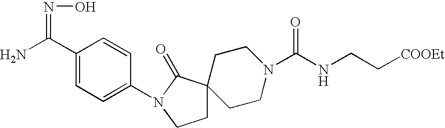

Similarly, other aspects of the invention, methods are provided for preventing or inhibiting tumor cell formation and for destroying a tumor in a subject in a subject comprising administering to the subject in need of such therapy a therapeutically effective amount of an activated α_{IIb}β₃ receptor antagonist.

The spiro compounds and other compounds are disclosed in U.S. Pat. Nos. 6,291,469 and 6,528,534, and related patents and patent applications. Methods of synthesizing the spiro compounds of the invention may be found in the many examples of U.S. Pat. Nos. 6,291,469 and 6,528,534 and related patents and patent applications. Methods of evaluating the spiro compounds for GPlb-lila inhibition and/or anti-metastatic activity are included in the examples of the present invention and methods provided in U.S. Pat. Nos. 6,291,469 and 6,528,534 and related patents and patent applications. Such methods include, but are not limited to, the following assay methods:

No. 1—the ELISA IIb-IIIa Assay:

In the following assay, GPIIb-IIIa may be prepared in purified form, by a method such as described by Fitzgerald, L. A., et al., Anal Biochem (1985) 151:169-177, (the disclosure of which is incorporated herein by reference in its entirety). GPIIb-IIIa is coated onto microtiter plates. The coated support is then contacted with fibrinogen and with the test material (e.g., spiro compounds of Formula I above) and incubated for a sufficient time to permit maximal binding of fibrinogen to the immobilized GPIIb-IIIa. Fibrinogen is typically provided at a concentration of about 5-50 nM and the test material can, if desired, be added at a series of dilution. Typical inicubations are 2 to 4 hours at 25° C., the time and temperature being interdependent.

After incubation, the solution containing the fibrinogen and test material is removed and the level of binding of fibrinogen measured by quantitating bound fibrinogen to GPIIb-IIIa. Any suitable means of detection may be used, but it is convenient to employ labeled fibrinogen, for example using biotinylated labels. Such methods are well known in the art.

A. Description of Assays—Plate Assays

Purified platelet GPIIb-IIIa receptor may be prepared as described by Fitzgerald, L. A., et al., Anal Biochem (1985) 151:169-177 (1985). Vitronectin receptor is prepared as described by Smith, J. W., J. Biol. Chem. (1988) 263:18726-18731. After purification, the receptors are stored in 0.1% Triton X-100 at 0.1-1.0 mg/ml.

The receptors are coated to the wells of 96-well flat-bottom ELISA plates (Linbro EIA-Plus microtiter plate, Flow Laboratories) after diluting 1:200 with a solution of 20 mM Tris-HCI, 150 mM NaCl, 1 mM CaCl₂, pH 7.4, to reduce the Triton X-100 concentration to below its critical micellar concentration and adding an aliquot of 100 μl to each well. The wells are all allowed to incubate overnight at 4° C., and then aspirated to dryness. Additional sites are blocked by the addition of bovine serum albumin (BSA) at 35 mg/ml in the above buffer for two hours at 30° C. to prevent nonspecific binding. The wells are then washed once with binding buffer (50 nM Tris-HCl, 100 mM NaCl 2 mM CaCl₂, 1 mg/ml BSA).

The corresponding ligands (fibrinogen, von Willebrand Factor, or vitronectin) are conjugated to biotin using commercially available reagents and standard protocols. The labeled ligands are added to the receptor-coated wells at final concentration of 10 nM (100 μl/well) and incubated for 3 hours at 25° C. in the presence or absence of the test samples. After incubation, the wells are aspirated to dryness and bound ligand is quantitated.

The bound protein is detected by the addition of anUbiotin antibody conjugated to alkaline phosphatase followed by addition of substrate (p-nitrophenyl phosphate), and determination of the optical density of each well at 405 nM. Decreased color development is observed in wells incubated with test samples which inhibit binding of ligand to receptor.

No. 2- The Platelet Aggregation Assay

In addition to the ELISA IIb-IIIa assay previously described, the Aggregation-Human PRP/ADP Assay is useful for evaluating therapeutic compounds. Platelet-rich plasma was prepared from healthy human volunteers for use in determining inhibition of platelet aggregation by the compounds. Blood was collected via a 21-gauge butterfly cannula, using a two-syringe technique into 1/10 volume of 3.8% trisodium citrate.

Platelet-rich plasma is prepared at room temperature by centrifugation of the citrated whole blood at 100×g for twelve minutes. The platelet rich plasma contained approximately 200-400,000 platelets//μl. Platelet-poor plasma is prepared by centrifugation of citrated whole blood at 12,000×g for 2 minutes. Platelet aggregation is assayed in a 4-channel platelet aggregation profiler (PAP-4, Biodata, Hatboro, Pa.) according to the manufacturers directions. Inhibition of platelet aggregation was analyzed by adding varying amounts of adenosine diphosphate (ADP) to stirred human platelet-rich plasma. Specifically, the human platelet-rich plasma is incubated with the compound being tested for 1 minute at 37° C. prior to the addition of a variety of aggregating agents most often ADP 5 μM, but also 1 μg/ml collagen, 1 μM U46619 and 0.3 μM platelet activating factor.

In another aspect of the invention, a method is provided for treating, preventing or inhibiting tumor cell metastasis to bone in a subject comprising replacing substantially all bone marrow affected by tumor cell metastasis transplant in the subject, wherein said bone marrow is replaced with β₃^{-/-} bone marrow. The Examples of the present invention present an animal model of bone metastases using genetic models of osteoclast function, e.g., the osteopetrotic src⁴mice and the osteosclerotic β₃^{-/-} mice. The protection from tumor osteolysis seen in the srce and β₃^{-/-} mice underscores that host osteoclasts are critical to tumor bone destruction and represent osteoclast-specific therapeutic anti-neoplastic targets. However, as shown in the Examples below, β₃^{-/-} mice were not only protected from tumor associated bone destruction, but also from migration of tumor cells to bone. β₃^{-/-} mice supported intramedullary tumor growth after direct intra-tibial (“IT”) tumor cell injection. However, after arterial LV tumor cell injection, few tumors entered the bone marrow cavity. Thus, transplantation of β₃^{-/-} bone marrow in mice protect the mice from tumor cell metastasis and tumor cell formation. In addition, transplantion of β₃^{-/-} bone marrow in mice destroys tumors in the bone of such mice.

Those of skill in the art will also recognize that the mouse model used in the present invention correlates to treatments and therapies in other mammals, including humans. Although the transplantation techniques described in the Examples section below would require modification for use in humans and other mammals, such modifications are within the skill of the art. Transplantation techniques for humans may be found in Sabiston, Textbook of Surgery, W.B. Saunders Company, Chapter 20, pp. 382-506 (1997). The in vitro β₃^{-/-} bone marrow cell culture described in the Examples section below could also be modified by those of skill in the art for culturing human and other mammalian β₃^{-/-} bone marrow for in vitro studies and transplantation into a human or other mammalian subject.

In a related aspect, β₃ integrin expression may be controlled through antisense or short interfering RNA (“siRNA”) methods which are well known in the art. For example, the β₃ integrin gene or RNA transcribed from the β₃ integrin gene may be targeted in a cell by introducing an oligonucleotide which hybridizes specifically to the β₃ integrin gene or transcribed RNA. In applications in humans, a portion of the human gene sequence could be targeted which is represented by GenBank Accession Number NM_{—}000212 which can be accessed on the Internet. In an antisense application, an oligonucleotide can be designed complementary to the gene or transcribed RNA which interferes with the translation to β₃ integrin. The encoded β₃ integrin would then not be fully expressed, and in some cases would not be expressed at all. Depending on the amount of the oligonucleotide delivered to the cell and other conditions which may be manipulated by methods known to those skilled in the art, the amount of β₃ integrin expressed may be carefully controlled within the cell and within a living organism.

Similarly, siRNAs may be designed to target β₃ integrin expression according to the Rational Design section below. Such siRNAs would interfere with β₃ integrin expression in a controlled manner. Regardless of the technique used to control the expression of β₃ integrin, those of skill in the art would recognize that the results would be similar to those observed in the β₃^{-/-} examples provided herein. Such techniques may involve delivering a composition into a cell. Numerous techniques have been developed for transfection of cells in vitro. The basic goal of all transfection techniques is to introduce the nucleic acid into a target cell. Transfection techniques may be broadly classified as either direct or indirect methods. Direct methods involve the manual introduction of nucleic acid. Examples of direct transfection include microinjection or microprojectile transfection. Indirect transfection techniques are numerous but can be broadly classified into viral transfection techniques, liposome transfection techniques and phagocytosis techniques.

Such techniques may be adapted by those of skill in the art for delivering a composition in vivo. This may include gene therapy techniques or targeted transfection/transduction of a particular organ system or organ, including the skeletal system, bone, and bone cells. Decreasing the expression in vivo may also comprise transforming a cell to express a polynucleotide anti-sense to at least a portion of an endogenous polynucleotide encoding β₃ integrin. Those of skill in the art will recognize many well known methods for accomplishing such transformation.

As described in the Examples below, the protection from bone metastases seen in the β₃^{-/-} mice was not mediated by microvasculature or other roles of β₃ integrins by demonstrating that protection from bone metastases could be transferred to recipient mice by bone marrow transplantation. Thus, the involvement of platelets provides a novel basis for treating, preventing or inhibiting tumor metastasis and tumor formation.

Pharmaceutical Preparations and Methods of Administration

The identified compositions treat, inhibit, control and/or prevent tumor cell metastasis, tumor growth and tumor destruction, particularly in bone, and can be administered to a subject at therapeutically effective doses for the inhibition, prevention, prophylaxis or therapy for such metastasis and growth. The compositions of the present invention comprise a therapeutically effective dosage of a spiro compound to antagonize the αIIbβ₃ receptor, a term which includes therapeutically, inhibitory, preventive and prophylactically effective doses of the compositions of the present invention and is more particularly defined below. Without being bound to any particular theory, applicants surmise that these pharmaceutical compositions prevent metastasis, tumor cell growth and destroy tumors when administered to a subject suffering from such a condition by limiting platelet interactions and aggregation. The subject is preferably an animal, including, but not limited to, mammals, reptiles and avians, more preferably horses, cows, dogs, cats, sheep, pigs, and chickens, and most preferably human.

Therapeutically Effective Dose

In addition to the dosage ranges disclosed in U.S. Pat. Nos. 6,291,469 and 6,528,534, toxicity and therapeutic efficacy of such compositions can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compositions that exhibit large therapeutic indices are preferred. While compositions exhibiting toxic side effects may be used, care should be taken to design a delivery system that targets such compositions to the site of affected tissue in order to minimize potential damage to uninfected cells and reduce side effects.

The data obtained from the cell culture assays and animal studies can be used in formulating a range of dosages for use in humans and other mammals. The dosage of such compositions lies preferably within a range of circulating plasma or other bodily fluid concentrations that include the ED₅₀with little or no toxicity. The dosage may vary within this range depending upon the dosage form employed and the route of administration utilized. For any composition used in the methods of the invention, the therapeutically effective dose can be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information can be used to more accurately determine useful doses in humans and other mammals. Composition levels in plasma may be measured, for example, by high performance liquid chromatography.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regime for treating a disease condition with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as part of a drug combination. Thus, the dosage regime actually employed may vary widely from subject to subject.

Formulations and Use

In addition to the formulations disclosed in U.S. Pat. Nos. 6,291,469 and 6,528,534, pharmaceutical compositions for use in accordance with the present invention may be formulated by known methods which include, but are not limited to, parenteral, oral, topical, intradermal, intramuscular, intraperitoneal, intravenous, subcutaneous, intranasal, epidural, and ophthalmic routes. The compositions may be administered by any convenient route, for example by infusion or bolus injection, by absorption through epithelial or mucocutaneous linings (e.g., oral mucosa, rectal and intestinal mucosa) and may be administered together with other biologically active agents. It is preferred that administration is localized, but administration may also be systemic.

The compositions may be formulated in any conventional manner using one or more pharmaceutically acceptable carriers or excipients. Thus, the compositions and their pharmaceutically acceptable salts and solvates may be specifically formulated for administration by inhalation or insufflation (either through the mouth or the nose) or oral, buccal, parenteral or rectal administration. Pharmaceutically acceptable compositions may take the form of charged, neutral and/or other pharmaceutically acceptable salt forms. Examples of suitable pharmaceutical carriers are described in REMINGTON'S PHARMACEUTICAL SCIENCES (A. R. Gennaro, Ed.), 20th edition, Williams & Wilkins Pa., USA (2000).

These compositions can take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

Parenteral Administration

The compositions may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The compositions may take such forms as suspensions, solutions or emulsions in oily or aqueous vehicles, and may contain formulatory agents such as suspending, stabilizing and/or dispersing agents.

Alternatively, the active ingredient may be in powder form for constitution with a suitable vehicle, such as sterile pyrogen-free water, before use. For example, a parenteral therapeutic composition may comprise a sterile isotonic saline solution containing between 0.1 percent and 90 percent weight per volume of the spiro compound. For example, a solution may contain from about 5 percent to about 20 percent, more preferably from about 5 percent to about 17 percent, more preferably from about 8 to about 14 percent, and most preferably about 10 percent spiro compound in solution (percent weight per volume). Where necessary, the composition may also include a solubilizing agent and a local anesthetic such as lignocaine to ease pain at the site of the injection.

Injectable preparations (e.g., sterile injectable aqueous or oleaginous suspensions) may be formulated according to the known art using suitable dispersing or wetting agents and suspending agents. The sterile injectable preparation may also be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the preparation of injectables.

Oral Administration

For oral administration, the pharmaceutical compositions may take the form of tablets or capsules prepared by conventional means with pharmaceutically acceptable excipients such as binding agents, fillers, lubricants and disintegrants:

A. Binding Agents
Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pa., USA). An exemplary suitable binder is a mixture of microcrystalline cellulose and sodium carboxymethyl cellulose sold as AVICEL RC-581 by FMC Corporation. The tablets may optionally be coated by methods well known in the art.

B. Fillers
Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

C. Lubricants
Lubricants include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof.

Additional lubricants include, for example, a syloid silica gel (AEROSIL 200, manufactured by W. R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

D. Disintegrants
Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.
   The binder/filler in pharmaceutical compositions of the invention is typically present in about 50 to about 99 weight percent of the pharmaceutical composition. Typically, about 0.5 to about 15 weight percent of disintegrant, preferably about 1 to about 5 weight percent of disintegrant, may be used in the pharmaceutical composition. A lubricant may optionally be added, typically in an amount of less than about 1 weight percent of the pharmaceutical composition. Specific examples of pharmaceutically-acceptable carriers and excipients that may be used to formulate oral dosage forms containing the compositions used in this invention, are described in U.S. Pat. Nos. 6,291,469 and 6,528,534. Techniques and compositions for making solid oral dosage forms are described in Marshall, “Solid Oral Dosage Forms,” Modern Pharmaceutics (Banker and Rhodes, Eds.), 7:359427 (1979). Other less typical formulations are known in the art.

Liquid preparations for oral administration may take the form of solutions, syrups or suspensions. Alternatively, the liquid preparations may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations may also contain buffer salts, flavoring, coloring, perfuming and sweetening agents as appropriate. Preparations for oral administration may be suitably formulated to give controlled release of the active composition. Oral formulations preferably contain 10% to 95% active ingredient. For buccal administration the compositions may take the form of tablets or lozenges formulated in a conventional manner.

Controlled- and Sustained-Release Administration

All controlled-release pharmaceutical products have a common goal of improving drug therapy over that achieved by their non-controlled counterparts. Ideally, the use of an optimally designed controlled-release preparation in medical treatment is characterized by a minimum of a composition being employed to prevent, treat, cure or control the condition in a minimum amount of time. Advantages of controlled-release formulations include:
1) extended activity of the composition; 2) reduced dosage frequency; and 3) increased patient compliance. In addition, controlled-release formulations can be used to effect the time of onset of action or other characteristics, such as blood levels of the composition, and thus may affect the occurrence of side effects.

Most controlled-release formulations are designed to initially release an amount of a composition that promptly produces the desired therapeutic effect, and gradually and continually release other amounts of the composition to maintain this level of therapeutic effect over an extended period of time. In order to maintain this constant level of a composition in the body, the composition must be released from the dosage form at a rate that will replace the amount of composition being metabolized and excreted from the body. The controlled-release of an active ingredient may be stimulated by various inducers, for example pH, temperature, enzymes, water, or other physiological conditions or compounds.

Controlled release systems may include, for example, an infusion pump which may be used to administer the composition in a manner similar to that used for delivering insulin or chemotherapy to specific organs or tumors. Typically, the composition is administered in combination with a biodegradable, biocompatible polymeric implant that releases the composition over a controlled period of time at a selected site. Examples of polymeric materials include polyanhydrides, polyorthoesters, polyglycolic acid, polylactic acid, polyethylene vinyl acetate, and copolymers and blends thereof. In addition, a controlled release system can be placed in proximity of a therapeutic target, thus requiring only a fraction of a systemic dose.

In addition to the common dosage forms set out above, the compounds of the invention may also be administered by controlled release means or delivery devices that are well known to those of ordinary skill in the art. Such devices and compositions can be used to provide slow or controlled-release of one or more of the active ingredients therein using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable controlled-release formulations known to those of ordinary skill in the art, including those described herein, may be readily selected for use with the pharmaceutical compositions of the invention. Thus, single unit dosage forms suitable for oral administration, such as tablets, capsules, gel caps, caplets, and the like, that are adapted for controlled-release are encompassed by the invention.

Inhalation Administration

The composition may also be administered directly to the lung by inhalation. For administration by inhalation, a composition may be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas may be used to deliver a composition directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device may be used to administer a composition to the lung. DPI devices typically use a mechanism such as a burst of gas to create a cloud of dry powder inside a container, which may then be inhaled by the patient. DPI devices are also well known in the art and may be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of the compound and a suitable powder base such as lactose or starch for these systems.

Another type of device that may be used to deliver a composition to the lung is a liquid spray device supplied, for example, by Aradigm Corporation. Liquid spray systems use extremely small nozzle holes to aerosolize liquid composition formulations that may then be directly inhaled into the lung.

In one exemplary embodiment, a nebulizer device is used to deliver a composition to the lung. Nebulizers create aerosols from liquid composition formulations by using, for example, ultrasonic energy to form fine particles that may be readily inhaled. Examples of nebulizers include devices supplied by Sheffield/Systemic Pulmonary Delivery Ltd., Aventis and Batelle Pulmonary Therapeutics.

In another exemplary embodiment, an electrohydrodynamic (“EHD”) aerosol device is used to deliver a composition to the lung. EHD aerosol devices use electrical energy to aerosolize liquid composition solutions or suspensions. The electrochemical properties of the composition formulation are important parameters to optimize when delivering this composition to the lung with an EHD aerosol device and such optimization is routinely performed by one of skill in the art. EHD aerosol devices may more efficiently deliver compositions to the lung than existing pulmonary delivery technologies. Other methods of intra-pulmonary delivery of compositions will be known to the skilled artisan and are within the scope of the invention.

Liquid composition formulations suitable for use with nebulizers and liquid spray devices and EHD aerosol devices will typically include the composition with a pharmaceutically acceptable carrier. In one exemplary embodiment, the pharmaceutically acceptable carrier is a liquid such as alcohol, water, polyethylene glycol or a perfluorocarbon. Optionally, another material may be added to alter the aerosol properties of the solution or suspension of the composition. For example, this material may be a liquid such as an alcohol, glycol, polyglycol or a fatty acid. Other methods of formulating liquid composition solutions or suspensions suitable for use in aerosol devices are known to those of skill in the art.

Depot Administration

The compositions may also be formulated as a depot preparation. Such long acting formulations may be administered by implantation (e.g., subcutaneously or intramuscularly) or by intramuscular injection. Thus, the compositions may be formulated with suitable polymeric or hydrophobic materials such as an emulsion in an acceptable oil or ion exchange resins, or as sparingly soluble derivatives such as a sparingly soluble salt. Other preparations for depot administration will be apparent to those of skill in the art.

Topical Administration

For topical application, the composition may be combined with a carrier so that an effective dosage is delivered, based on the desired activity ranging from an effective dosage, for example, of 1.0 μM to 1.0 mM. In one embodiment, a topical composition is applied to the skin. The carrier may be in the form of, for example, and not by way of limitation, an ointment, cream, gel, paste, foam, aerosol, suppository, pad or gelled stick. A topical form may also consist of a therapeutically effective amount of the composition in an ophthalmologically acceptable excipient such as buffered saline, mineral oil, vegetable oils such as corn or arachis oil, petroleum jelly, Miglyol 182, alcohol solutions, or liposomes or liposome-like products. Any of these compositions may also include preservatives, antioxidants, antibiotics, immunosuppressants, and other biologically or pharmaceutically effective agents which do not exert a detrimental effect on the composition. Other preparations for topical administration will be apparent to those of skill in the art.

Suppository Administration

The compositions may also be formulated in rectal compositions such as suppositories or retention enemas containing conventional suppository bases such as cocoa butter or other glycerides and binders and carriers such as triglycerides, microcrystalline cellulose, gum tragacanth or gelatin. Suppositories generally contain an active ingredient in the range of 0.5% to 10% by weight. Other preparations for suppository administration will be apparent to those of skill in the art.

Other Systems of Administration

Various other delivery systems are known in the art and can be used to administer the compositions of the invention. Moreover, these and other delivery systems may be combined and/or modified to optimize the administration of the identified compositions.

Packaging

The compositions may, if desired, be presented in a pack or dispenser device which may contain one or more unit dosage forms containing the composition. The pack may for example comprise metal or plastic foil, such as a blister pack. The pack or dispenser device may be accompanied by instructions for administration.

Rational siRNA design for RNA interference

Short-interfering RNAs suppress gene expression through a highly regulated enzyme-mediated process called RNA interference (RNAi). RNAI involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial efficient RNAi. Reynolds et al. provide methods for identifying such features. A. Reynolds et al., “Rational siRNA design for RNA interference”, Nature Biotechnology 22(3), March 2004. In that study, eight characteristics associated with siRNA functionality were identified: low GIC content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection.

To address the question of what determines siRNA functionality, Reynolds et aL performed a comprehensive analysis of a panel of 180 siRNAs targeting every other position of two 197-base regions of firefly luciferase and human cyclophilin B mRNA (90 siRNAs per gene). Previous work had correlated low G/C content within target mRNA regions with efficient siRNA silencing. To extend this observation, the 197-base regions were chosen to avoid extended G/C stretches enabling identification of other factors important for siRNA functionality. The 180 duplexes exhibited widely varying silencing abilities, showing that a two-base shift in target position was sufficient to significantly alter siRNA functionality. These results suggest that functionality is determined by the siRNA-specific properties, and not by the local mRNA target properties. This distinguishes RNAi from antisense, where silencing is dependent on antisense target site accessibility and is determined by the local mRNA conformation. About 78% of the siRNAs induced more than 50% silencing (>F50) of the two mRNA regions targeted. As expected, the probability of selecting highly potent siRNAs, that is, duplexes capable of more than 95% gene silencing (>F95) was low. Of all the duplexes, 24.4% targeting firefly luciferase and 11.1% targeting human cyclophilin B, silenced their respective targets by 95% or greater.

To quantify correlations between functionality and other biophysical or thermodynamic properties, the G/C content was calculated for each duplex and the functional classes of siRNAs were re-sorted accordingly (<F50, >F50, >F80, >F95). Most highly functional siRNAs (F95) had a G/C content that ranged between 36% and 52%. The G/C content groups bracketing the 36-52% group contained an increased proportion of nonfunctional siRNAs. Thus, a 30-52% G/C content was selected as criterion I for siRNA functionality, consistent with previous findings. Application of this criterion alone provided only a marginal advantage for selecting functional siRNAs from the panel: selection of F50 and F95 siRNAs was improved by 3.2% and 1.2%, respectively (P=0.0386).

Next, low internal stability of the siRNA at the 5′ antisense end was established as a quantifiable criterion. The A/U base pair (bp) content is a relatively simple measure of local internal stability; therefore, the frequency of A/U bp was determined for each of the five terminal positions of the duplex (5′ sense/5′ antisense) across the entire panel. Duplexes were categorized by the number of A/U bp in positions 1-5 and 15-19 of the sense strand. The thermodynamic flexibility of the duplex 5′-end (positions 1-5, sense strand) did not appear to correlate with silencing potency, whereas that of the 3′-end (positions 15-19, sense strand) correlated well with efficient silencing. In addition, no duplexes lacking A/U bp in positions 15 -19 were functional, whereas the presence of one or more A/U bp in this region conferred increasing degrees of functionality. As a result, the occurrence of three or more A/U bp defined criterion II. Although more significant than the G/C content criterion I, the increase in functional siRNA selection was only marginal upon application of criterion II: a 5.7% and 3.6% increase for F50 and F95 duplexes, respectively (P=0.0128). siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (Tₘ ). Sequences with high Tₘ values would favor internal hairpin structures. Sorting the functional siRNA classes by Tₘ revealed that duplexes lacking stable internal repeats were better silencers (no F95 duplexes exhibited Tₘ>60° C. or predicted hairpin structures). In contrast, about 56.5% of the duplexes having Tₘ values <20° C. were F80. Thus, high internal repeat stability is inversely proportional to silencing and defines criterion III (predicted hairpin structure; Tₘ<20° C.; P=0.0016). To summarize, analysis of thermodynamic characteristics of the 180 siRNA test panel revealed three criteria important for siRNA functionality: moderate to low G/C content, low internal stability of the sense 3′-end (5′ antisense) and a lack of internal repeats.

To evaluate the contributions of specific, sequence-related determinants, the siRNA test panel was re-sorted into functional (F80) and nonfunctional (<F50) subsets. The following positive and negative discriminants were selected to further evaluate their relative contributions to functionality (sense strand position): an A at positions 3 and 19, a U at position 10, the absence of G at position β₃ and the absence of G or C at position 19. When evaluated for the presence of an A at position 19 of the sense strand, selection of nonfunctional duplexes decreased from 22.2% to 16.2%, and selection of F95 duplexes increased from 17.8% to 25% (P=0.0029). Thus, an A at this position defined criterion IV. When evaluated for the presence of an A in position 3 of the sense strand, 26.6% of the duplexes were F95, compared with 17.8% randomly selected siRNAs (P=0.0723). The presence of a U in position 10 of the sense strand was observed in 30.6% of the F95 duplexes selected by this attribute (P=0.0531). These properties became criterion V and VI, respectively. Two negative sequence-related criteria were also identified. The absence of a G or C at position 19 of the sense strand and a G at position 13 of the sense strand correlated with selecting functional duplexes (P=0.00007 and P=0.0026, respectively). These were defined as criteria VII and VII.

Application of each criterion individually provided marginal but significant increases in the probability of selecting a potent siRNA. To further improve selection, all eight criteria were combined into an algorithm subsequently used to evaluate the siRNA test panel as in Table 1.

TABLE 1
Functional class distribution of siRNAs for each criterion

| | | | | Relative |
|---|---|---|---|---|
| | | Functional | | change from |
| Criterion | | group | siRNAs (%) | random (%) |
| I. | 30%-52% G/C content | <F50 | 19.0 | −3.2 |
| | | ≧F50 | 81.0 | 3.2 |
| | | ≧F80 | 55.5 | 3.8 |
| | | ≧F95 | 19.0 | 1.2 |
| II. | At least 3 ‘A/U’ | <F50 | 16.5 | −5.7 |
| | bases at positions 15-19 | ≧F50 | 83.5 | 5.7 |
| | (sense strand) | ≧F80 | 59.2 | 7.6 |
| | | ≧F95 | 21.4 | 3.6 |
| III. | Absence of internal | <F50 | 18.5 | −3.7 |
| | repeats (Tₘ of potential | ≧F50 | 81.5 | 3.7 |
| | internal hairpin | ≧F80 | 56.5 | 4.8 |
| | is Tₘ < 20° C.) | ≧F95 | 19.4 | 1.6 |
| IV. | An ‘A’ base at | <F50 | 16.2 | −6.0 |
| | position 19 (sense strand) | ≧F50 | 83.8 | 6.0 |
| | | ≧F80 | 70.6 | 18.9 |
| | | ≧F95 | 25.0 | 7.2 |
| V. | An ‘A’ base at | <F50 | 20.3 | −1.9 |
| | position 3 (sense strand) | ≧F50 | 79.7 | 1.9 |
| | | ≧F80 | 57.8 | 6.1 |
| | | ≧F95 | 26.6 | 8.8 |
| VI. | A ‘U’ base at | <F50 | 16.7 | −5.6 |
| | position 10 (sense strand) | ≧F50 | 83.3 | 5.6 |
| | | ≧F80 | 63.9 | 12.2 |
| | | ≧F95 | 30.6 | 12.8 |
| VII. | A base other than | <F50 | 14.6 | −7.6 |
| | ‘G’ or ‘C’ at position 19 | ≧F50 | 85.4 | 7.6 |
| | (sense strand) | ≧F80 | 66.0 | 14.4 |
| | | ≧F95 | 21.4 | 3.6 |
| VIII. | A base other than | <F50 | 17.4 | −4.8 |
| | ‘G’ at position 13 | ≧F50 | 82.6 | 4.8 |
| | (sense strand) | ≧F80 | 57.6 | 5.9 |
| | | ≧F95 | 21.2 | 3.4 |

Each siRNA was assigned a score according to the following logic: satisfaction of criteria I, III, IV, V, VI earned one point. Failure to satisfy the negative criteria (criteria VII and VII) resulted in a one-point decrease. For criterion II, one point was added for each A or U base present in positions 15-19 (potential 5 points total). Sorting the panel by score revealed that most duplexes earning 6 points or more were functional whereas most nonfunctional siRNAs attained scores of 5 to −1. Among duplexes with a score of 6, 17% were nonfunctional whereas 100% of those scoring −1 were <F50. As a result, a score of 6 defined the cutoff for selecting siRNAs. All duplexes scoring higher than 6 (15.5% of the panel) comprised the ‘selected’ group and the remaining duplexes (84.5% of the panel) were ‘eliminated’ (P=0.000018). Of note, some ‘eliminated’ siRNAs were functional, suggesting that subsequent identification of additional discriminants and incorporation into the algorithm would improve functional duplex selection. Nevertheless, the current algorithm was quite effective in identifying functional duplexes from this siRNA panel. Of the duplexes selected, all exhibited potency >F50. Among the selected group, 92.9% were F80, of which 46.4% were F95.

After validation of the algorithm on a set of target sequences, the algorithm improved the average probability of selecting an F50 siRNA from 46.5% (random selection) to 96.6% in this validation set. Furthermore, approximately 56.7% of the rationally selected siRNAs were F80, compared to 16.3% for randomly selected siRNAs. Thus, on average, the algorithm ensured functional duplex selection for all six genes with over a 3.5-fold improvement in achieving 80% knockdown. Of note, the percentage of randomly selected F80 duplexes within the validation set is substantially lower than within the initial test set. This bias towards functional siRNAs within the test set may be the result of predefining the target region for low G/C context based on previous recommendations.

In addition, the validation set includes targets that are less amenable to silencing where few (e.g., PLK) or none (e.g., DBI) of the random duplexes were functional. The factors described here may be predictive of functional associations important for each step in RNAi. For example, complementary strands with internal repeats favor stable hairpin structures thus decreasing the effective concentration of the functional duplex and correlating negatively with siRNA functionality. This study also supports previous observations that siRNA functionality correlates with overall low internal stability of the duplex and low internal stability of the sense 3′ end; both attributes are thought to promote strand selection and entry into the RISC. Interestingly, siRNAs with very high or very low overall stability are less likely to be functional. Presumably, high internal stability prevents efficient unwinding whereas very low stability might reduce siRNA target affinity and subsequent mRNA cleavage by the RISC.

The remaining five criteria describe base preferences at specific positions and are very intriguing when considering their potential roles in target recognition and mRNA cleavage. Base preferences for A (but not C or G) at position 19 of the sense strand are particularly interesting because they reflect the same bias observed for naturally occurring microRNA precursors. That is, 75% of the reported microRNA precursor sequences contain a U at position 1 (corresponding to A in position 19 of the sense strand of siRNAs) whereas G was underrepresented in this same position. This supports the hypothesis that both microRNA precursors and siRNA duplexes are processed by similar if not identical protein machinery. As previously suggested, reference for an A/U bp at the 5′ antisense closing position reflects a requirement for the efficient and selective strand entry into the RISC for both siRNAs and microRNA precursors.

Among the sequence-specific criteria, the preference for U at position 10 of the sense strand exhibited the greatest impact, enhancing selection of an F95 siRNA by 12.8%. Activated RISC preferentially cleaves target mRNA between nucleotides 10 and 11 relative to the 5′ end of the complementary targeting strand. Therefore, RISC, like most endonucleases, prefers to cleave 3′ of U rather than A, G or to a lesser extent, C. By performing a comprehensive analysis of an siRNA test panel, we successfully identified at least eight specific determinants of siRNA functionality. Applied individually, each determinant alone is not sufficient to ensure silencing. However, an algorithm integrating all eight factors substantially enhanced functional siRNA identification (that is, 29 out of 30 rationally designed siRNAs induced more than 50% silencing of six different targets).

This study for the rational design of siRNA in silico revealed previously unidentified features correlating with siRNA functionality. These attributes most likely reflect potential biophysical and molecular interactions that occur during RNAi. The rational design algorithm derived from the eight criteria described here represents a subset of all factors that contribute to efficient interactions and would be improved with the larger data set. Further analyses of functional and nonfunctional siRNA sequences and of the RNAi mechanism will refine the role and significance of existing criteria and define new factors, thus improving the reliability of siRNA rational design for functional genomic analysis. However, rational siRNA design rmay be accomplished by applying the algorithm to the sequences of the present invention and optimized according to the methods of Reynolds et al.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

The Role of β₃ Integrin in Cancer Metastasis and Tumor Formation

In these Examples, C57B6/129 β₃^{-/-} mice were created at Washington University, St. Louis, Mo. C57B6 mice (Harlan Laboratories, Indianapolis, Ind.) were used for the antagonism experiments. src^{-/-} mice (C57B6/129) were obtained from Jackson Laboratories, Bar Harbor, Me. All mice were bred and maintained with sterilized food, water and bedding. B16-F10 mouse melanoma cells (a gift from Dr. David Fisher, Dana Farber Cancer Institute, Boston, Mass.) were cultured in DMEM with 10% Fetal Bovine Serum. After 0.1% trypsin/0.2% EDTA treatment, B16 cells were resuspended in phosphate-buffered saline (PBS) prior to in vivo injection. Cell viability was determined by trypan blue exclusion. Osteoclasts were formed from bone marrow derived macrophages in α-MEM media containing 10% FCS, 100 ng/mL GST- RANKL (created at Washington University, St. Louis, Mo.) and murine M-CSF (10 ng/mL, R+D, Minneapolis, Mo). Multinucleated OCs were identified by TRAP staining (Sigma-Aldrich, St. Louis, Mo.).

The role of β₃ integrin was analyzed in the development of osteolytic tumor invasion in 6-8 week-old β₃^{+/+}, β₃^{+/-} and β₃^{-/-} mice injected with B16F10 murine melanoma cells via the cardiac left ventricle (LV). The overall LV injection procedure-related survival rate in 6-12 week old β₃^{-/-} mice was diminished at 59% compared to 93% in β₃^{+/+}, and 91% in β₃^{+/-} mice, most likely due to bleeding secondary to the defect in platelet function in these mice. The β₃^{-/-} mice that survived the procedure had no subsequent mortality from bleeding. The β₃^{+/+} mice injected with 1×10⁵ B16 cells LV had a mean survival (Kaplan Meier method) of 16 days (data not shown), displaying cachexia (>20% weight loss), labored breathing from chest and lung tumors and paraplegia from vertebral body tumor invasion and spinal cord compression. Therefore an end-point of 14 days post LV injection was chosen to evaluate bone metastases.

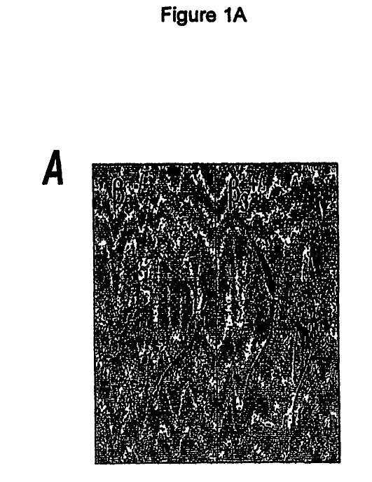

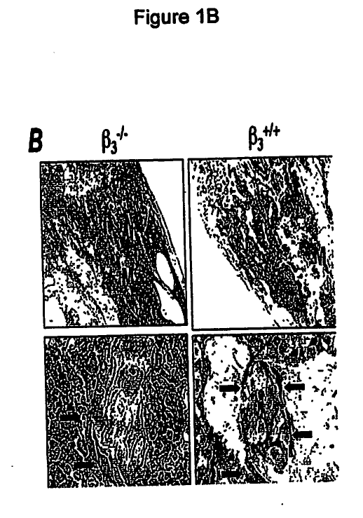

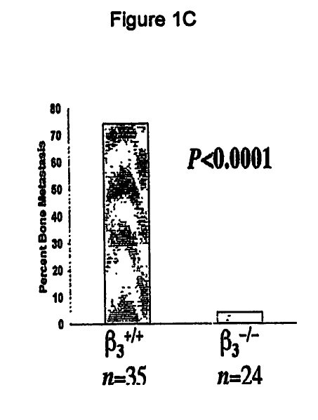

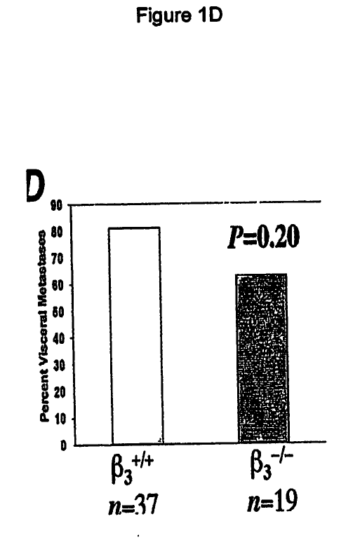

FIG. 1 analysis discloses that β₃^{-/-} mice are protected from osteolytic bone metastases. 14 days after B16 LV injection, visible pigmented bone lesions were recorded (FIG. 1a), followed by histological confirmation of tumors and associated bone loss in the femurs and tibias (FIG. 1b). The visible pigmented B16 melanoma cells bone metastases of FIG. 1a were seen in β₃^{+/+} but not in β₃^{-/-} mice, 14 days after B16 tumor cell LV injection. Pathologic fracture developed in β₃^{+/+} femur. FIG. 1b shows a TRAP-stained femur cross section of a B16 LV injected β^{+/+} mouse (4×, 40× objectives) Pigmented B16 cells (T) are growing throughout the bone marrow (M). Tumor associated osteolysis induced fracture (F) of bone cortex. Arrows mark TRAP positive osteoclasts recruited to B16 tumor cells within the bone matrix in β₃^{+/+} mice. No B16 cells were evident in β₃^{-/-} femurs in 23/24 mice. Histological data of FIG. 1b also provided evidence for bone destruction in the long bones of β₃^{+/+} mice, severe enough in some places to result in disruption of the bone cortex. In the β₃^{+/+} mice, 74% (26/35) developed bone metastases by day 14 (FIG. 1c). FIG. 1c depicts the percent of mice with bone metastases in femur and tibia 14 days after LV injection of B16 cells for β₃^{+/+} compared to β₃^{-/-} mice. (P<0.0001 Fisher exact t-test). Heterozygote β₃^{+/-} mice developed bone metastases with similar frequency as the β₃^{+/+}. In contrast only 1 out of 24 of the β₃^{-/-} mice injected had histological evidence of bone metastases in the femur and tibia (FIG. 1c) while the remaining 23 β₃^{-/-} mice had no B16 tumors seen in the bone marrow. Visceral metastases were evaluated to confirm that tumor cells successfully entered the arterial system. 81% of the β₃^{+/+} mice had visceral metastases compared to a rate of 63% of the β₃^{-/-} mice (P=0.20) (FIG. 1d). FIG. 1d shows the percent of mice with pigmented visceral metastases (in liver, lung, adrenal, gut, or brain) 14 days after LV injection of B16 melanoma cells in β₃^{+/+} compared to β₃^{-/-} mice (P=0.2 by Fisher exact t-test). (e) B16osteolytic bone invasion in β₃^{+/+} mice. The sites of visceral metastases (mesentery, adrenal, intestine, kidney, skin, liver, and brain) were not different in the β₃^{+/+} and β₃^{-/-} mice. β₃^{-/-} mice are therefore selectively protected from bone metastasis.

The inhibition of the bone metastasis seen in the β₃^{-/-} mice reflect a bone microenvironment unable to support tumor cell growth. B16 cells were inoculated or saline directly into the tibial cavity of the β₃^{+/+} and β₃^{-/-} mice to provide an example of such inhibition. Fourteen days later, histomorphometry of the tumor cell invasion showed induced osteolysis.

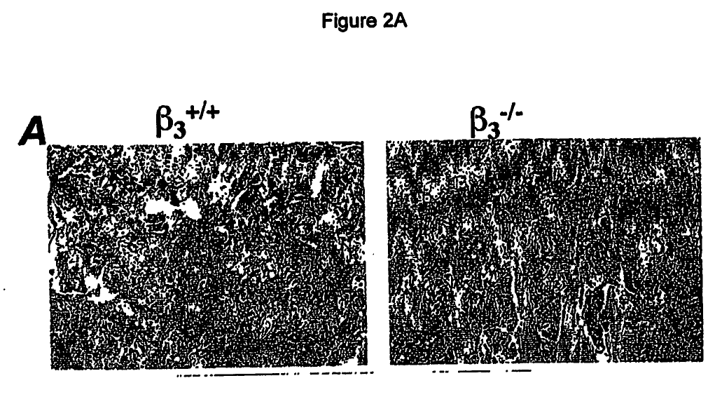

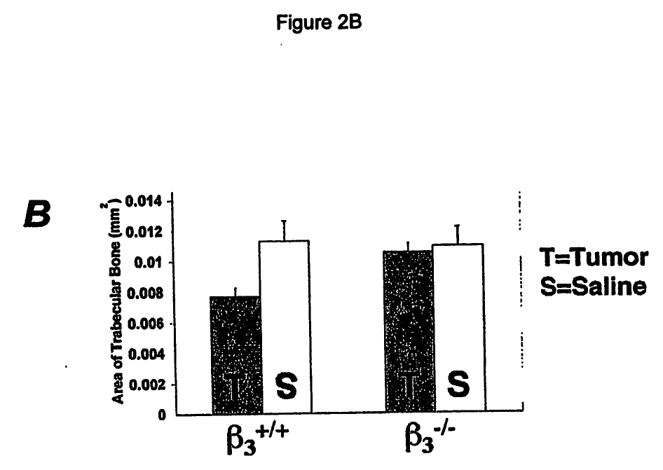

FIG. 2 analysis discloses that β₃^{-/-} mice are protected from osteolytic bone invasion after direct inoculation of tumor cells into the bone marrow cavity by intra-tibial injection. The examples of FIG. 2 used 10⁴ B16 cells injected into the left tibia. Saline was injected into the right tibia as internal control. Tumor cells proliferated to fill the marrow cavities of both the β₃^{-/-} and β₃^{+/+} mice (FIG. 2a). FIG. 2a shows TRAP/Hematoxalin staining of tibial cross section 14 days after B16 or saline IT injection. B16 IT injected β₃^{+/+} tibia on the left with marked trabecular bone loss and complete replacement of marrow cavity with tumor, compared to B16 IT injected β₃^{-/-} tibia on the right with marrow replacement by pigmented tumor cells but little associated trabecular bone destruction. B16 cells induced significant trabecular bone destruction in the β₃^{+/+} tibia compared to saline injection (P<0.01), whereas there was no significant difference in the trabecular bone area between B16 and saline injected β₃^{-/-} tibia (FIG. 2b). FIG. 2b provides histomorphometric analysis of trabecular bone area for saline (S) and B16 (T) IT injected tibia (each data point is a compilation of 12 equivalent tibial cross section measurements taken from 4 mice). B16 cells induced significant trabecular bone destruction in the β₃^{+/+} tibia compared to saline injection (P<0.01 measured by Paired t test), whereas there was no significant difference in the trabecular bone area between B16 and saline injected β₃^{-/-} tibia. Comparison of trabecular bone area between B16 injected β₃^{-/-} and β₃^{+/+} demonstrates protection from tumor associated bone destruction in the β₃^{-/-} mice (P<0.01 using Two sample t-test). In addition, FIG. 2c shows a Trabecular Bone Area 14 days after B16 intra-tibial injection. In this example, B16 injection has no affect on trabecular bone area in β₃^{-/-} marrow. These data show that B16 melanoma cells cannot induce bone destruction in the absence of β₃^{-/-} in host cells.

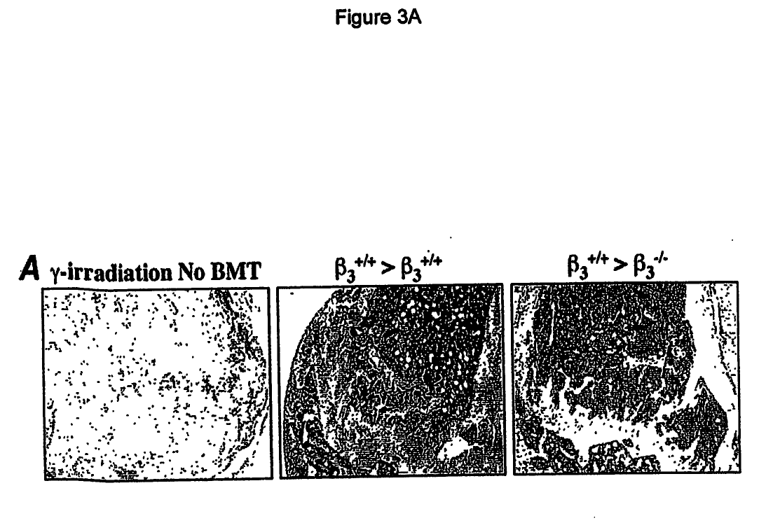

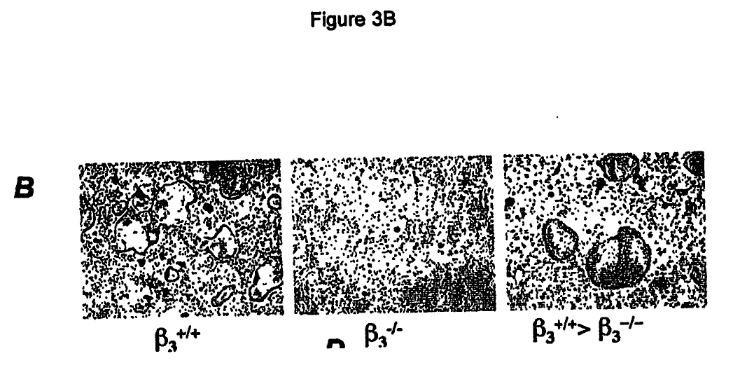

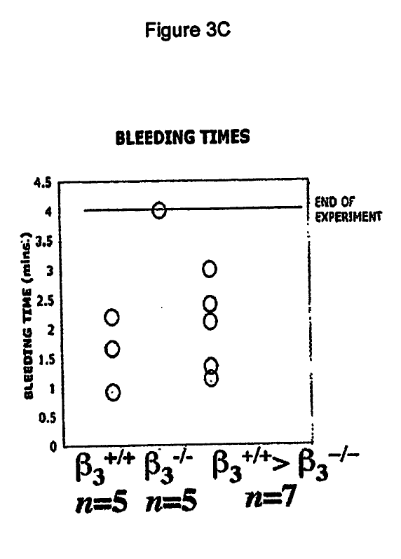

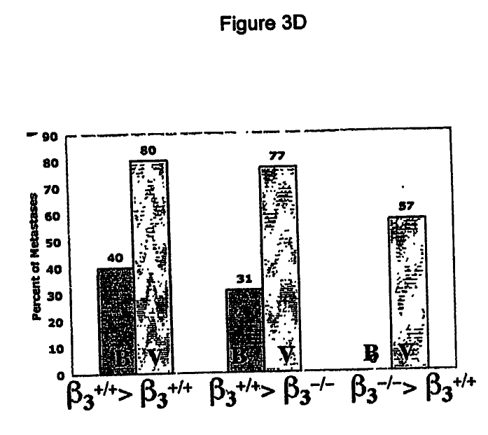

Osteoclasts and platelets are formed in the bone marrow. Bone marrow transplantation (BMT) was performed showing a protective effect from bone metastases observed in the β₃^{-/-} mice. In this example, 17β₃^{-/-} mice were transplanted with wild-type β₃^{+/+} bone marrow 24 hours after lethal irradiation (β₃^{+/+}<β₃^{-/-}). FIG. 3 analysis discloses that bone marrow transplantation (BMT) of β₃^{31 /-} marrow confers protection from osteolytic metastases. Recovery of the hematopoietic compartment 21 days after BMT was demonstrated by recovery of TRAP+osteoclasts in the marrow (FIG. 3a), recovery of osteoclasts with β₃^{+/+} phenotype (FIG. 3b) and recovery of platelet aggregation with normalization of bleeding times (FIG. 3c). FIG. 3a shows TRAP staining of femur 10 days after 950 rads gamma-irradiation in untransplanted control mouse demonstrating fatty marrow devoid of red marrow cells and loss of TRAP+ osteoclasts at growth plate. Recovery of TRAP+ osteoclasts is seen at growth plates of femurs 10 days after transplantation of β₃^{+/+} marrow into β₃^{+/+} positive control (middle panel) or into β₃^{-/-} mouse (right panel). FIG. 3b shows in vitro TRAP staining of cultured osteoclasts result in multinucleated β₃+/+osteoclasts with well-formed actin rings, compared to β₃^{-/-} osteoclasts at day 5. BMT with β₃^{30 /+} marrow into β₃^{-/-} mice restores osteoclast with β3^{30 /+} phenotype (last panel) confirming osteoclast engraftment 3 weeks after lethal irradiation and bone marrow transplantation. FIG. 3c provides bleeding times performed on β₃^{-/-} mice 3 weeks after transplantation with β₃^{+/+} marrow confirm platelet engraftment and rescue of bleeding defect with BMT (5 mice/group not shown).

21 days after BMT, 1×10⁵ B16 cells were injected via LV. Fourteen days later the mice were examined for visual and histological evidence of B16 bone invasion. Four of 13 mice (31%) developed B16 bone invasion (FIG. 3d). More particularly, FIG. 3d shows the percentage of transplanted mice with visible bone metastases (B) and visceral metastases (V) 14 days after LV injection of B16 cells. β₃^{+/+}>β₃^{-/-} is positive control β₃^{+/+} marrow transplanted into β₃^{30 /+} mouse (n=1 0). β₃^{+/+}>β_{-/-} is β₃^{+/+} marrow transplanted into β₃^{-/-} animals (n=13) demonstrating that β₃^{30 /+} marrow can restore ability of B16 to induce bone metastases in β₃^{-/-} mice. β₃^{-/-}>β₃^{+/+} marrow transplanted into β₃^{30 /+} mice (n=7) demonstrates that β₃^{-/-} bone marrow can protect wild type mice from the bone metastases susceptibility as compared to β₃^{30 /+} mice (P=0.0004 using the Fisher exact t-test). Ten mice (77%) had visceral metastases confirming successful arterial delivery of the tumor cells (FIG. 3d). Thus the protection from bone metastases in β₃^{-/-} mice was partially disrupted after transplantation of β₃^{+/+} marrow.

One explanation for the lower incidence of bone metastases seen in the β₃^{-/-} mice reconstituted with WT marrow (31% vs. 74% in β₃^{30 /+} mice) is that the irradiation used for the bone marrow transplant inhibited tumor cell growth in bone. To determine the effect of irradiation, 10β₃^{30 /+} mice were lethally irradiated and transplanted β₃^{+/+} bone marrow β₃^{+/+}>β₃^{+/+}) as a control. 40% of these mice developed bone metastases and 80% had visceral metastases, comparable to the rate seen in β₃^{30 /+}>β₃^{-/-})mice (FIG. 3d).

To determine whether the protection from bone metastases in the β₃^{-/-} mice might also be influenced by an abnormal bone architecture, β₃^{-/-} bone marrow was transplanted into β₃^{+/+} mice (β₃^{-/-}>β₃^{+/+}). Survival rate from the LV tumor cell injection was similar to unirradiated β₃^{-/-} mice, with only 7/16 mice (44%) surviving the LV procedure, indicating the presence of β₃^{-/-} platelets. Importantly all 7β₃^{-/-}β₃^{+/+} mice were protected from B16 osteolytic bone metastases while four of the mice developed visceral metastases (FIG. 3d). Thus, the susceptibility to bone metastasis is transplantable and thereby lies in the hematopoietic compartment as depicted in FIG. 3e where bone metastases are shown to be significantly reduced in KO mice. Additionally, in FIG. 3f, bone marrow transplantation is shown to restore bone metastases in β₃^{-/-} mice. As seen from this figure, bone metastases are significantly diminished in bone transplanted mice.

Osteoclast defective mouse mutant, the src^{-/-} mouse, was utilized to validate that defective osteoclasts in the β₃^{-/-} mice are responsible for the protection from bone metastases independent of abnormal platelet function. The src^{-/-} mice have no reported defects in platelet aggregation, but develop osteopetrosis (lack of tooth eruption, markedly diminished marrow cavities, stunted growth) secondary to defective osteoclast bone resorption. src^{-/-} mice and src^{+/+} liftermates were LV injected with 1×10⁵ B16 cells.

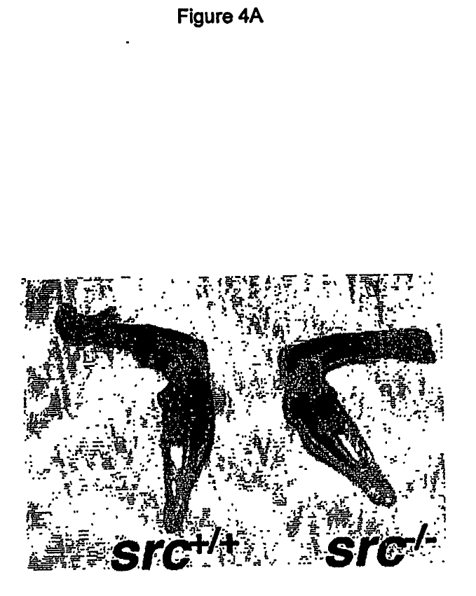

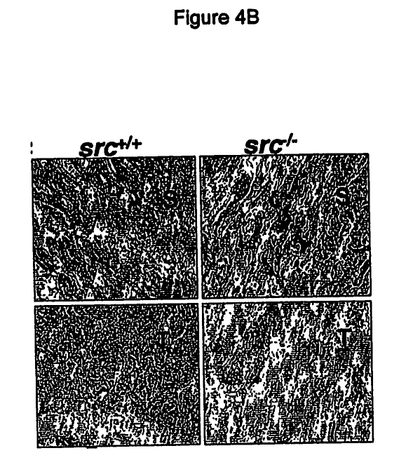

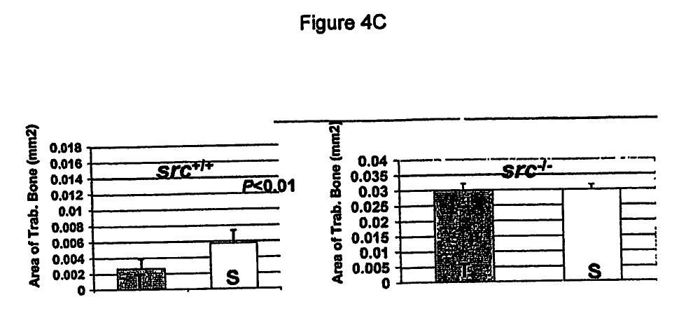

FIG. 4 depicts that osteoclast defective src^{-/-} mutant mice develop bone lesions without tumor associated bone destruction. All of the src mice survived the LV injection procedure, and had normal bleeding times (1-3 minutes, n=5 mice), confirming normal physiologic platelet aggregation in the src^{-/-} mice. 86% (6/7 mice) src^{-/-} mice and 75% (3/4 mice) src^{+/+} wild type littermates developed wide spread visible pigmented bone lesions after LV injection (FIG. 4a). FIG. 4a shows visible pigmented B16 melanoma cells bone lesions were seen in src^{+/+} and src^{-/-} mice 14 days after B16 tumor cell LV injection. Similar rates of visceral metastases were observed in 86% of the src^{-/-} mice and 75% of the src^{+/+mice. Thus the OC-defective src}^{-/-} was not protected from B16 tumor entry into bone, despite the severe osteopetrosis and diminished marrow space.

The B16 LV injected into src^{-/-} mice induced little trabecular bone destruction, despite tumor dissemination throughout the marrow cavity (FIGS. 4 b,c). FIG. 4b depicts the histology of TRAP-stained tibias from saline LV injected src^{+/+} and src^{-/-} mice compared to B16 LV injected mice. src^{-/-} bones have significantly more trabecular and cortical bone with a limited bone marrow cavity. B16 cells proliferate to fill available marrow space in both src^{-/-} and src ^{-/-} tibia. FIG. 4c depicts the histomorphometry results show tumor-induced trabecular bone loss in src^{+/+} mice compared to saline injected mice (P<0.01 by Two sample t-test). src^{-/-} mice injected with B16 cells show no decrease in bone volume compared to src^{-/-} saline injected controls. The src^{+/+} wild type littermates had B16 associated bone destruction compared to saline injected controls (P<0.01) (FIG. 4c). Because there was little bone destruction in B16 LV injected src^{-/-} mice and in B16 IT injected β₃^{-/-} mice (FIG. 4c), it can be deduced that the B16 cells require functional osteoclasts to induce tumor osteolysis.

These data underscore the point that the protection from bone metastases seen in the β₃^{-/-} mice and reversed by a bone marrow transplant is not solely explained by lack of proper osteoclast activation and resorption. However, the protection from tumor cells entering the bones of β₃^{-/-} mice, not seen in the src^{-/-} mice, is possibly caused by another transplantable hematopoietic cell, the platelet.

An activated α_{IIb}β₃ receptor antagonist in β₃^{+/+} mice was utilized in examples showing the role of platelet β₃ integrin in the protection from bone metastases seen in the β₃^{-/-} mouse. ML464 is an oral murine activated α_{IIb}β₃ receptor antagonist spiro compound with a serum half-life of 3 hours that inhibited platelet aggregation within 30 minutes of in vivo administration (measured by aggregometry from PRP of 5 mice). ML464 is represented by the following formula: 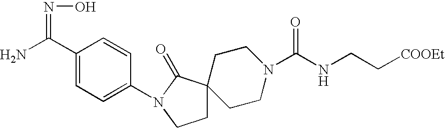
ML728, the ML464 active metabolite, is represented by the following formula: 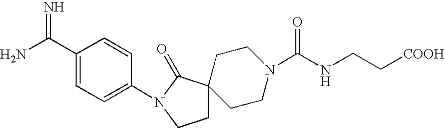
Both ML464 and ML728 are preferable spiro compounds of the present invention. However, those skilled in the art will recognize that additional activated α_{IIb}β₃ receptor antagonists may be used in the methods, the efficacies of which may be determined using the protocols of the present disclosure. Additional protocols are available to those skilled in the art to determine the efficacy of other activated α_{IIb}β₃ receptor antagonists. Such activated αIIb₃ receptor antagonists are meant to be encompassed within the scope of the present invention.

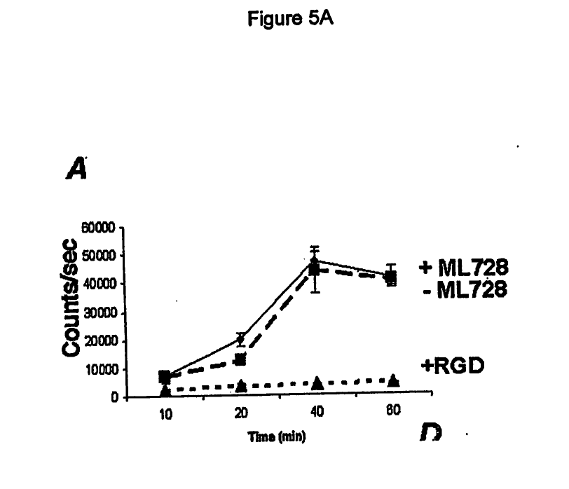

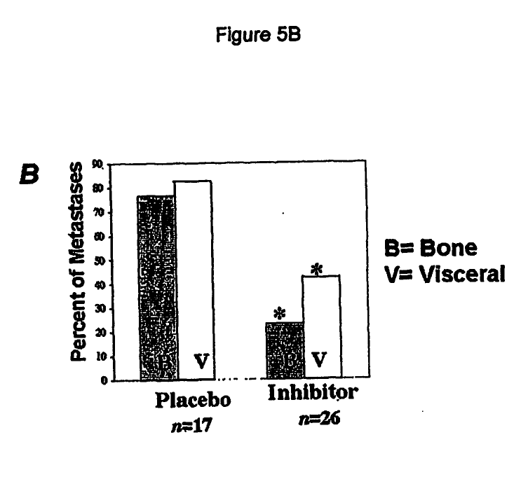

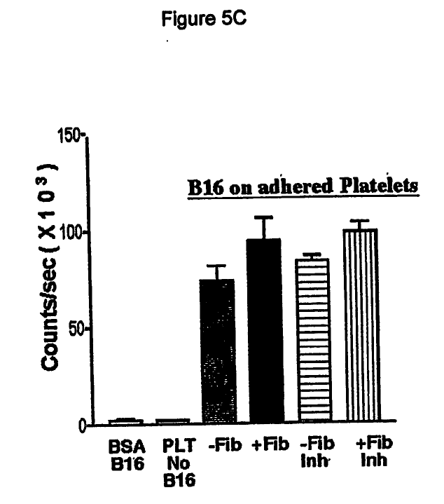

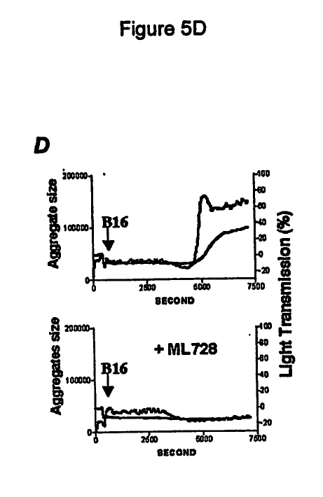

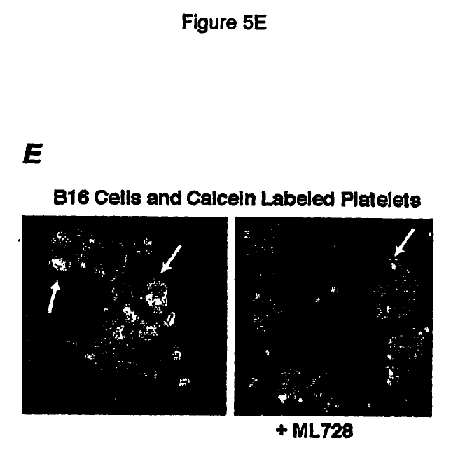

FIG. 5 analysis discloses that α_{IIb}β₃ Antagonist Reduces Metastases in β₃^{+/+} Mice. Platelet aggregation was disrupted for up to 10 hours after an oral dose of 100 mg/kg. To confirm that ML728, the active metabolite of ML464, does not inhibit mouse α_{V}β₃ function, adhesion of B16 cells to α_{V}β₃ ligand fibrinogen coated surface in the presence and absence of ML728 was examined. B16 cells express αᵥβ₃ but not α_{IIb}β₃ (data not shown). Adhesion of B16 cells to fibrinogen coated surface was not inhibited by 37.5 μM ML728 while 1 mM linear RGD peptide completely blocked adhesion (FIG. 5a) demonstrating that α_{V}β₃ function is not inhibited by ML728. FIG. 5a shows B16 cells spreading on fibrinogen coated surface (diamond), was not inhibited by 37.5 μM ML728 (square) but completely inhibited by RGD peptide (triangle). 37.5 μM was the peak plasma concentration level of the active metabolite measured in mice 30 minutes after oral administration of ML464.

The activated α_{IIb}β₃ receptor antagonist was tested based on its ability to diminish metastases in wild type mice. Activated α_{IIb}β₃ receptor antagonist, ML464, was administered to 40β₃^{+/+} mice by oral gavage at a dose of 100 mg/Kg. The mice were treated 30 minutes prior to the LV injection of 1×10⁵ B16 cells, and then every 12 hours for 5 doses (2.5 days of treatment out of the 14 day experiment). 14/40 (35%) mice died from bleeding complications after the LV injection indicative of effective disruption of α_{IIb}β₃ mediated platelet aggregation. 1/17 (6%) placebo treated mice died after the LV injection procedure. The surviving inhibitor treated mice had significantly less bone metastases (23%) compared to the vehicle-dosed littermates (76%) (P=0.0013) (FIG. 5b). FIG. 5b shows ML464 (an activated α_{IIb}β₃ receptor antagonist and platelet binding inhibitor) administered to wild type β₃^{+/+} mice) 30 minutes prior to B16 LV injection and then every 12 hours for 2.5 days. Placebo in DMSO carrier was also administered by oral gavage. Mice were evaluated 14 days after B16 injection for bone and visceral metastases. Percentage of mice with bone (B) or visceral (V) metastases placebo treated mice (n=17) and ML464 treated mice (n=26). The asterisks (*) highlights that metastases decreased in ML464 treated mice compared to placebo treated mice (P=0.0013 for bone and P=0.0125 for visceral using Fisher's Exact t-test).

A decrease in visceral metastases between placebo and ML464 treated mice was also observed (P=0.0125). Furthermore it was observed that the ML464 treated mice that did develop metastases had reduced number and size of visceral metastases compared to the placebo group; however, tumor sizes were not measured using this assay, but only the presence or absence of metastases.

B16 melanoma cells can stimulate platelet aggregation. In FIG. 5C, B16 cells were added to stirred washed mouse platelets. Platelet microaggregate and large aggregates were formed six minutes after addition of B16 cells but not after buffer or COS-7 cells were added (data not shown). FIG. 5c shows B16 cells added to unactivated platelets induce platelet aggregation as measured in an aggregometer. Arrow represents the addition of B16 cells to platelets. Blue line represents microaggregates and black line is total aggregates (micro and large). Addition of 5 μM ML728, an α_{llb}₃ receptor antagonist to stirred platelets prior to addition of B16 cells completely inhibited platelet aggregation.

B16 stimulated platelet aggregation was inhibited by 5 μM ML728, the active metabolite of the oral α_{IIb}β₃ receptor antagonist, ML464 (FIG. 5c, lower panel). The IC₅₀ for inhibition of B16 stimulated platelet aggregation was 1 μM. ML728 inhibited platelet-tumor interaction, shown in an example utilizing calcein labeled washed platelets incubated with B16 cells. Aggregated platelets bound tumor cells and enhanced crosslinking of tumor cells (FIG. 5d left panel). FIG. 5d shows calcein labeled fluorescent mouse platelets adhere to unlabeled B16 tumor cells (arrows) and form aggregates of platelets and tumor cells (left panel). Addition of 5 μM ML728 inhibited tumor cell and platelet aggregation/clumping but not platelet-tumor cell adhesion (right panel).

Addition of ML728 inhibited platelet aggregation and platelet aggregate mediated crosslinking of B16 cells; however, ML728 did not inhibit platelet binding to tumor cells (FIG. 5d right panel). In a separate assay, ML728 did not prevent B16 adhesion to spread platelets even in the presence of fibrinogen (FIG. 5e). FIG. 5e shows B16 cells adhered to spread platelets in the presence (+Fib) or absence (−Fib) of fibrinogen, which was not inhibited by ML728 α_{IIb}β₃ receptor antagonist/binding inhibitor (Inh). Platelets alone (PLT no B16) and B16 cells on BSA coated surface (B16 BSA) served as controls. Thus α_{llb}β₃ inhibition by ML728 does not interfere with B16 adhesion to platelets but does effectively block tumor associated platelet-platelet aggregation in vitro. Thus, an activated α_{IIb}β₃ receptor antagonist protected mice from bone and visceral metastasis. Further aspects of the protocols used in the present Examples are provided below.

Bone histology and Histomorphometry

Mouse femurs and tibias were excised, cleaned of soft tissue, fixed in formalin and decalcified in 14% EDTA. Long bones were embedded in paraffin and sliced at equivalent sections coronally through the centre of the bone. Histological sections were stained with hematoxylin and eosin and for tartrate-resistant acid phosphatase (TRAP) activity. Trabecular bone area was measured according to standard protocol using the Osteomeasure Analysis System (Osteometrics Incorporated, Decatur, Ga).

Bone Metastases

Mice were anaesthetized followed by injection with 30 gauge needles of 1×10⁵ B16 cells in 100 μl PBS into the left cardiac ventricle as previously described. Confirmation of correct insertion was established by pulsing geyser of blood in the syringe. After the injection mice were monitored daily for 14 days.

Intratibial Injection

30 gauge needles were used to inject 1×10⁴ B16 cells or PBS control in 50 μl into the tibia in anaesthetized mice. The knee was flexed and the needle inserted into the tibia, boring the needle through the epiphysis and epiphyseal plate for delivery of the cells into the metaphysis. Mice were monitored daily for tumor growth.

Bone marrow transplants (BMT)

Mice were irradiated with 950 rads of gamma-radiation. The mice were transplanted with 5×10⁶ whole bone marrow cells from β₃^{+/+} or β₃^{-/-} mice via tail vein injections within 24 hours of lethal irradiation. Three weeks after BMT after normalization of blood counts, and bleeding times, mice were LV injected with B16 cells.

α_{IIb}β₃ Receptor Analysis

C57BI6 mice were used to analyze metastasis antagonism. ML464, an oral spiro compound α_{IIb}β₃ receptor antagonist containing the 2,8-diaza-spiro[4,5]deca-1 -one nucleus (Millennium Pharmaceuticals, Boston, Mass.). The active metabolite ML728 of the pro-drug ML464 has a serum half-life of β₃ hours. Blockade of platelet aggregation is complete at 30 minutes after oral gavage but persists for up to 10 hours. 100 mg/kg of ML464 or placebo was administered every 12 hours via oral-gavage for 5 doses. Thirty minutes after the first oral-gavage dose, mice were LV injected with B16 cells and evaluated for metastases 14 days later.

Tumor cell induced platelet aggregation

Mouse blood was drawn into 4U/ml of Heparin and centrifuged at 200 g for 20 min to obtain platelet rich plasma (PRP). PRP was centrifuged at 1500 g for 10 min and platelets were washed in CGS buffer (3 mM trisodium citrate, 120 mM sodium chloride and 30 mM dextrose pH 7.0) and resuspended in Hepes-Tyrodes buffer (12 mM Sodium bicarbonate, 138 mM sodium chloride, 5.5 mM glucose, 2.9 mM potassium chloride, 10 mM Hepes pH 7.4) containing 1 mM CaCl₂ and MgCl₂. Washed mouse platelets at 2×10⁸/ml were aggregated with 1×10⁸/ml of B16 cells in the presence or absence of ML728, an active metabolite of an oral α_{IIb}β₃ antagonist ML464. Calcein AM (Molecular probes, Eugene, Oreg.) labeled washed platelets (2×10⁷/ml) were incubated with 1×10⁶ B16 cells/ml in the presence or absence of 5 μM ML728.

B16 Adhesion to Spread Platelets

2.4×10⁷ platelets/ml were allowed to spread in 96 well plates for 1 hour at 37° C. Wells were blocked with 2.5% BSA for 30 min at 37° C. 8X1 0 4 B16 cells/well were added in the presence or absence of 1 μM fibrinogen and 37.5 μM ML728. Adherent cells were lysed, stained with DNA dye and read in the fluorescence reader according to the manufacturer's instructions (Cyquant kit, Molecular Probes, CA).

Melanoma Cell Adhesion to Fibrinogen

After 0.05% trypsin/EDTA treatment, B16 cells were resuspended in serum free DMEM containing 25 μg/ml soybean trypsin inhibitor, washed and plated in fibrinogen coated wells in the presence or absence of ML728 (37.5 μM) or linear RGD peptide (1 mM). Adherent cells were measured using the Cyquant kit.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which does not depart from the spirit or scope of the present inventive discovery. Such modifications are also intended to fall within the scope of the appended claims.

### REFERENCES CITED

Incorporated herein by reference in their entirety include:

1. Mundy, G. R. Metastasis to bone: causes, consequences and therapeutic opportunities. Nat Rev Cancer 2, 584-93 (2002).

2. Gasic, G. J., Gasic, T. B. & Stewart, C. C. Antimetastatic effects associated with platelet reduction. Proc NatI Acad Sci U S A 61, 46-52 (1968).

3. Pearlstein, E., Ambrogio, C. & Karpatkin, S. Effect of antiplatelet antibody on the development of pulmonary metastases following injection of CT26 colon adenocarcinoma, Lewis lung carcinoma, and B16 amelanotic melanoma tumor cells into mice. Cancer Res 44, 3884-7 (1984).

4. Karpatkin, S., Pearistein, E., Ambrogio, C. & Coller, B.S. Role of adhesive proteins in platelet tumor interaction in vitro and metastasis formation in vivo. J Clin Invest 81, 1012-9 (1988).

5. Felding-Habermann, B., Habermann, R., Saldivar, E. & Ruggeri, Z. M. Role of beta3 integrins in melanoma cell adhesion to activated platelets under flow. J Biol Chem 271, 5892-900 (1996).

6. Francis, J. L. & Amirkhosravi, A. Effect of antihemostatic agents on experimental tumor dissemination. Semin Thromb Hemost 28, 29-38 (2002).

7. Amirkhosravi, A., Amaya, M. & Siddiqui, F. A. Blockade of Gpilb/llia inhibits the release of vascular endothelial growth factor (VEGF) from tumor cell-activated platelets and experimental metastasis. Platelets 10, 285-292 (1999).

8. Felding-Habermann, B. et al. Integrin activation controls metastasis in human breast cancer. Proc Natl Acad Sci U S A 98, 1853-8 (2001).

9. Clohisy, D. R. & Ramnaraine, M. L. Osteoclasts are required for bone tumors to grow and destroy bone. J Orthop Res 16, 660-6 (1998).

10. Guise, T. A. et al. Evidence for a causal role of parathyroid hormone-related protein in the pathogenesis of human breast cancer-mediated osteolysis. J Clin Invest 98, 1544-9 (1996).

11. Lipton, A. et al. Pamidronate prevents skeletal complications and is effective palliative treatment in women with breast carcinoma and osteolytic bone metastases: long term follow-up of two randomized, placebo-controlled trials. Cancer 88, 1082-90 (2000).

12. Yoneda, T. et al. Actions of bisphosphonate on bone metastasis in animal models of breast carcinoma. Cancer 88, 2979-88 (2000).

13. Rosen, L. S. Efficacy and safety of zoledronic acid in the treatment of bone metastases associated with lung cancer and other solid tumors. Semin Oncol 29, 28-32 (2002).

14. Saad, F. Treatment of bone complications in advanced prostate cancer: rationale for bisphosphonate use and results of a phase III trial with zoledronic acid. Semin Oncol 29,19-27 (2002).

15. Hynes, R. O. Integrins: versatility, modulation, and signaling in cell adhesion. Cell 69, 11-25 (1992).

16. Hood, J. D. & Cheresh, D. A. Role of integrins in cell invasion and migration. Nat Rev Cancer 2, 91-100 (2002).

17. Teti, A., Migliaccio, S. & Baron, R. The role of the alphαᵥbeta3 integrin in the development of osteolytic bone metastases: a pharmacological target for alternative therapy? Calcif Tissue Int 71, 293-9 (2002).

18. Phillips, D. R., Charo, I. F. & Scarborough, R. M. GPIIb-IIIa: the responsive integrin. Cell 65, 359-62 (1991).

19. Law, D. A. et al. Integrin cytoplasmic tyrosine motif is required for outside-in alphIIbbeta3 signalling and platelet function. Nature 401, 808-11 (1999).

20. Chen, Y. P. et al. Ser-752−−>Pro mutation in the cytoplasmic domain of integrin beta 3 subunit and defective activation of platelet integrin alpha IIb beta 3 (glycoprotein IIb-IIIa) in a variant of Glanzmann thrombasthenia. Proc Natl Acad Sci U S A 89, 10169-73 (1992).

21. Brooks, P. C., Clark, R. A. & Cheresh, D. A. Requirement of vascular integrin alpha v beta 3 for angiogenesis. Science 264, 569-71 (1994).

22. Horton, M. A., Taylor, M. L., Amett, T. R. & Helfrich, M. H. Arg-Gly-Asp (RGD) peptides and the anti-vitronectin receptor antibody 23C6 inhibit dentine resorption and cell spreading by osteoclasts. Exp Cell Res 195, 368-75 (1991).

23. Ross, F. P. et al. Interactions between the bone matrix proteins osteopontin and bone sialoprotein and the osteoclast integrin alpha v beta 3 potentiate bone resorption. J Biol Chem 268, 9901-7 (1993).

24. Engelman, V. W. A peptidomimetic antagonists of the avb3 integrin inhibits bone resorption in vitro and prevents osteoporosis in vivo. Journal of Clinical Investigation 99, 2284-2292 (1997).

25. Rodan, S. B. & Rodan, G. A. Integrin function in osteoclasts. J Endocrinol 154 Suppl, S47-56 (1997).

26. Inoue, M., Namba, N., Chappel, J., Teitelbaum, S. L. & Ross, F. P. Granulocyte macrophage-colony stimulating factor reciprocally regulates alphav-associated integrins on murine osteoclast precursors. Mol Endocrinol 12, 1955-62 (1998).

27. Hodivala-Dilke, K. M. et al. Beta3-huintegrin-deficient mice are a model for Glanzmann thrombasthenia showing placental defects and reduced survival. J Clin Invest 103, 229-38 (1999).

28. McHugh, K. P. Mice lacking b3 integrins are osteosclerotic because of dysfunctional osteoclasts. Journal of Clinical Investigation 105, 433-440 (2000).

29. Feng, X. et al. A Glanzmann's mutation in beta 3 integrin specifically impairs osteoclast function. J Clin Invest 107, 1137-44 (2001).

30. Arguello, F., Baggs, R. B. & Frantz, C. N. A murine model of experimental metastasis to bone and bone marrow. Cancer Res 48, 6876-81 (1988).

31. Soriano, P., Montgomery, C., Geske, R. & Bradley, A. Targeted disruption of the c-src proto-oncogene leads to osteopetrosis in mice. Cell 64, 693-702 (1991).

32. Tanaka, S. et al. c-Cbl is downstream of c-Src in a signalling pathway necessary for bone resorption. Nature 383, 528-31 (1996).

33. Ichinohe, T. et al. Collagen-stimulated activation of Syk but not c-Src is severely compromised in human platelets lacking membrane glycoprotein VI. J Biol Chem 272, 63-8 (1997).

34. Wu, Y. et al. Differential activation and redistribution of c-Src and Fyn in platelets, assessed by MoAb specific for C-terminal tyrosine-dephosphorylated c-Src and Fyn. Biochim Biophys Acta 1497, 27-36 (2000).

35. Staiano, N. et al. Echistatin inhibits the adhesion of murine melanoma cells to extracellular matrix components. Biochem Mol Biol Int 35, 11-9 (1995).

36. Silletti, S., Kessler, T., Goldberg, J., Boger, D. L. & Cheresh, D. A. Disruption of matrix metalloproteinase 2 binding to integrin alpha vbeta 3 by an organic molecule inhibits angiogenesis and tumor growth in vivo. Proc Natl Acad Sci U S A 98,119-24 (2001).

37. Borsig, L., Wong, R., Hynes, R. O., Varki, N. M. & Varki, A. Synergistic effects of L- and P-selectin in facilitating tumor metastasis can involve non-mucin ligands and implicate leukocytes as enhancers of metastasis. Proc Natl Acad Sci U S A 99, 2193-8 (2002).

38. Honore, P. et al. Osteoprotegerin blocks bone cancer-induced skeletal destruction, skeletal pain and pain-related neurochemical reorganization of the spinal cord. Nat Med 6, 521-8 (2000).

39. Coleman, R. E. Future directions in the treatment and prevention of bone metastases. Am J Clin Oncol 25, S32-8 (2002).

40. Body, J. J. et al. A phase I study of AMGN-0007, a recombinant osteoprotegerin construct, in patients with multiple myeloma or breast carcinoma related bone metastases. Cancer 97, 887-92 (2003).

41. Zhang, J. et al. Osteoprotegerin inhibits prostate cancer-induced osteoclastogenesis and prevents prostate tumor growth in the bone. J Clin Invest 107, 1235-44 (2001).

42. Kaplan, I. D., Valdagni, R., Cox, R. S. & Bagshaw, M. A. Reduction of spinal metastases after preemptive irradiation in prostatic cancer. International Journal of Radiation and Oncological Biology and Physiology 18, 1019-1025 (1990).

43. Reynolds, L. E. et al. Enhanced pathological angiogenesis in mice lacking beta3 integrin or beta3 and beta5 integrins. Nat Med 8, 27-34 (2002).

44. Weilbaecher, K. N. et al. Linkage of M-CSF signaling to Miff, TFE3, and the osteoclast defect in Mitf(mi/mi) mice. Mol Cell 8, 749-58 (2001).

45. Parfitt, A. M. Bone histomorphometry: Standardization of nomenclature, symbols, and units. Report of the ASBMR histomorphometry of nomenclature committee. Journal of Bone Mineral Research 2, 595-610 (1987).

46. Bakewell S J, et al., Platelet and osteoclast beta3 integrins are critical for bone metastasis. Proc Natl Acad Sci USA (2003 Nov 25) 100(24):14205-10.

What is claimed is:

1. A method for treating, preventing or inhibiting tumor cell metastasis in a subject comprising administering to the subject in need of such therapy a therapeutically effective amount of an activated α_{llb}β₃ receptor antagonist.

2. The method of claim 1, wherein the tumor cell metastasis targets an organ system of the subject.

3. The method of claim 2, wherein the tumor cell metastasis targets a skeletal system of the subject.

4. The method of claim 3, wherein the tumor cell metastasis targets a bone of the subject skeletal system.

5. The method of claim 3, wherein the tumor cell metastasis targets a bone cell of the subject skeletal system.

6. The method of claim 1, wherein the antagonist is a platelet-specific activated α_{IIb}β₃ receptor antagonist.

7. The method of claim 1, wherein the platelet-specific activated α_{IIb}β₃ receptor antagonist is a spiro compound.

8. The method of claim 7, wherein the spiro compound is represented by the formula:
Q-(L)_{Z}—Z—R₃
wherein
Z is a spirocyclic nucleus selected from the group consisting of Nucleus (A), (B), (C), or (D) represented by the formulas: 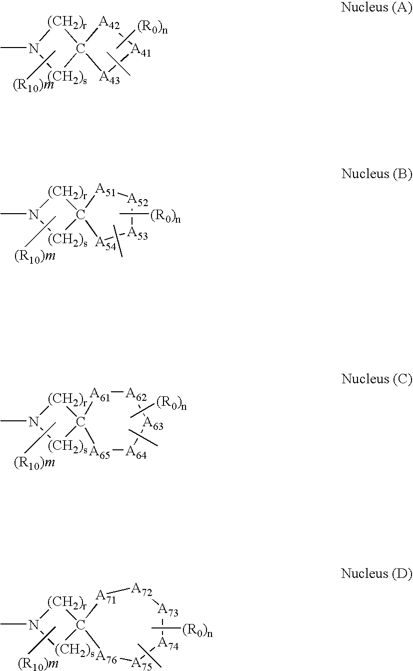
wherein
the group Q-(L)_{Z}- is bound to the nitrogen containing ring of nuclei (A), (B), (C), or (D) and the group R₃ is bound to the ring formed by the groups A₄₁, A₄₂, A₄₃, A₅₁, A₅₂, A_{53, A}₅₄, A₆₁, A₆₂, A₆₃, A₆₄, A₆₅, A₇₁, A₇₂, A₇₃, A₇₄, A₇₅, or A₇₆; or
the group R₃ is bound to the nitrogen containing ring and the group Q-(L)_{Z}- is bound to the ring formed by the groups A₄₁, A₄₂, A₄₃, A₅₁, A₅₂, A₅₃, A₅₄, A₆₂, A₆₃, A₆₄, A₆₅, A₇₁, A₇₂, A₇₃, A₇₄, A₇₅, or A₇₆;
r and s are independently a number from zero to 5 with the proviso that not both r or s are 0 and (r+s) is not more than 6, and z is zero or one;
atoms A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆ are independently selected from carbon, nitrogen, oxygen or sulfur, provided that at least one of said atoms is carbon;
provided that the hydrogens of the nitrogen containing part of the spirocycle Z may be substituted by a number of m substituents R₁₀, wherein;
m is a number from zero to (r+s); and
R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₁₀ may be ═══O or ═══S;
n is a number from zero to β₃ in Z of having nuclei (A), or a number from zero to 4 in Z having nuclei (B), a number from zero to 5 in Z having nuclei (C), or a number from zero to 6 in Z having nuclei (D);
R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₀ may be ═══O or ═══S; and
-(L)- is a bond or a divalent substituted or unsubstituted chain of from 1 to 10 atoms selected from the group consisting of carbon, nitrogen, sulfur, and oxygen;
Q is a basic group containing one or more basic radicals; and
R₃ is an acidic group containing one or more acid radicals;
or a pharmaceutically acceptable salt, solvate or pro-drug thereof.

9. The method of claim 7, wherein the spiro compound is represented by the formula: 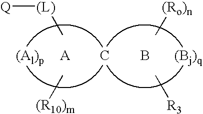
wherein
atoms Aᵢ and Bⱼ are independently selected from carbon, nitrogen, oxygen or sulfur, provided that at least one atom of Aᵢ is carbon, and at least one atom Bⱼ is carbon;
optionally, the rings of the spirobicycle formed by Aᵢ and Bⱼ , respectively, are partly unsaturated;
p and q are Independently numbers from 2 to 6;
m is a number from zero to p;
R₁₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₁₀ may be ═══O or ═══S, if p is 2 or one or two R₁₀ may be ═══O or ═══S, if p is a number from β₃ to 6;
n is the number from zero to q;
R₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₀ may be ═══O or ═══S, if q is 2 or one or two R₀ may be ═══O or ═══S, if q is a number from β₃ to 6;
-(L)- is a bond or a divalent substituted or unsubstituted chain of from 1 to 10 atoms selected from the group consisting of carbon, nitrogen, sulfur, and oxygen;
Q is a basic group containing one or more basic radicals; and
R₃ is an acidic group containing one or more acid radicals;
or a pharmaceutically-acceptable salt, solvate or pro-drug thereof.

10. The method of claim 7, wherein the spiro compound is represented by the formula: 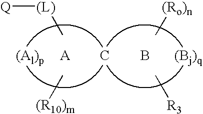
wherein
the spirocycle having (Aᵢ)ₚ, C, and (Bⱼ )_{q} is 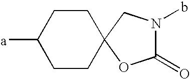
m is a number from zero to 9;
R₁₀is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;
n is a number from zero to 2;
R₀ is the same or different and is a non-interfering substituent Independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;
wherein Q-(L) is attached at a, and R₃ is attached at b;
-(L)- is a bond or a substituted or unsubstituted chain selected from the group consisting of CO, CO(C₁-C₆ alkyl), O(C₁-C₆ alkyl), NHCO, and C₁-C₆ alkyl;
Q is a basic group selected from the group consisting of amino, imino, amidino, hydroxyamidino, N-alkylamidine, N,N′-dialkylamidine, N-arylamidine, aminomethyleneamino, aminomethylamino, guanidino, aminoguanidino, alkylamino, dialkylamino, trialkylamino, alkylideneamino, pyrrolyl, imidazolyl, pyrazolyl, pyridyl, pyrazinyl, pyrimidinyl, indolizinyl, isoindolyl, 3H-indolyl, indolyl, 1H-indazolyl, purinyl, 4H-quinolizinyl, isoquinolyl, quinolyl, phthalazinyl, naphthyridinyl, quinoxalinyl, quinazolinyl, cinnolinyl, amide, thioamide, benzamidino, pteridinyl, 4aH-carbozolyl, carbozolyl, beta-carbolinyl, phenanthridinyl, acridinyl, phenanthrolinyl, phenazinyl, phenarsazinyl, phenothiazinyl, pyrrolinyl, imidazolidinyl, imidazolinyl, pyrazolidinyl, pyrazolinyl, piperidyl, piperazinyl, indolinyl, isoindolinyl quinuclidinyl, morpholinyl, any of the foregoing radicals substituted on a benzene ring, optionally substituted with R_{2c} , wherein R_{2c} is hydrogen or halogen and any of the foregoing radicals substituted by amino, imino, amidino, hydroxyamidino, aminomethyleneamino, iminomethylamino, guanidino, alkylamino, dialkylamino, trialkylamino, tetrahydroisoquinoline, dihydrosioindole, alkylideneamino or 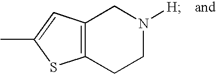
R₃ is an acidic group selected from the group consisting of CO₂ R₅, (C₁-C₆ alkyl)CO₂ R₅, CO(C₁-C₆ alkyl)CO₂ R₅, CONH(C₁-C₆ alkyl)CO₂ R₅, (C₁-C₆ alkyl)CH(NHR₄)CO₂ R₅, CO(C₁-C₆ alkyl)CH(NHR₄)CO₂ R₅, or CONH(C₁-C₆ alkyl)CH(NHR₄)CO₂ R₅, wherein R₄ is SO₂ (C₁-C₆ alkyl), SO₂ aryl, or SO₂ (substituted aryl); and
R₅ is hydrogen, C₁-C₆ alkyl, aryl, or substituted aryl;
or a pharmaceutically acceptable salt, solvate or pro-drug thereof.

11. The method of claim 7, wherein the spiro compound is represented by the formula: 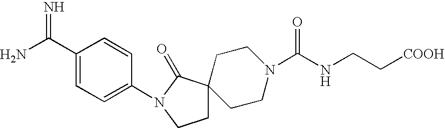
or a pro-drug thereof.

12. The method of claim 11, wherein the pro-drug is represented by the formula:

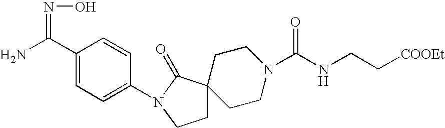

13. A method for preventing or inhibiting tumor cell formation in a subject comprising administering to the subject in need of such therapy a therapeutically effective amount of an activated α_{IIb}β₃ receptor antagonist.

14. The method of claim 13, wherein the tumor cell the tumor cell is formed in an organ system of the subject.

15. The method of claim 14, wherein the tumor cell the tumor cell is formed in a skeletal system of the subject.

16. The method of claim 15, wherein the tumor cell the tumor cell is formed in a bone of the subject skeletal system.

17. The method of claim 15, wherein the tumor cell the tumor cell is formed in a bone cell of the subject skeletal system.

18. The method of claim 13, wherein the antagonist is a platelet-specific activated α_{llb}β₃ receptor antagonist.

19. The method of claim 18, wherein the platelet-specific activated α_{IIb}β₃ receptor antagonist is a spiro compound.

20. The method of claim 19, wherein the spiro compound is represented by the formula:
Q-(L)_{Z}—R₃
wherein
Z is a spirocyclic nucleus selected from the group consisting of Nucleus (A), (B), (C), or (D) represented by the formulas: 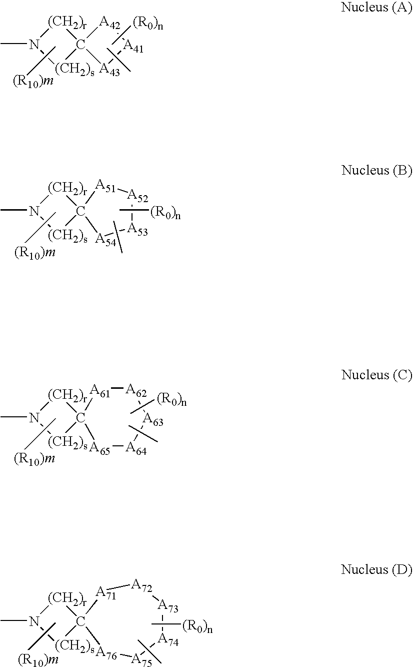
wherein
the group Q-(L)_{Z}- is bound to the nitrogen containing ring of nuclei (A), (B), (C), or (D) and the group R₃ is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆; or
the group R₃ is bound to the nitrogen containing ring and the group Q-(L)_{Z}- is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆;
r and a are independently a number from zero to 5 with the proviso that not both r or a are 0 and (r+s) is not more than 6, and z is zero or one;
atoms A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆ are independently selected from carbon, nitrogen, oxygen or sulfur, provided that at least one of said atoms is carbon;
the hydrogens of the nitrogen containing part of the spirocycle Z may be substituted by a number of m substituents R₁₀, wherein;
m is a number from zero to (r+s); and
R₁₀ is the same or different and is a non-interfering substituent Independently selected from alkyl, halosubstituted alkyl, alkenyl, alkynyl, cycloalkyl, aryl, arylalkyl, hydroxy, alkoxy, arylaikoxy, amino, substituted amino, carbamoyl, carboxy, acyl, cyano, halo, nitro, sulfo, ═══O, or ═══S, with the proviso that only one or two R₁₀ may be ═══0 or ═══S;
n is a number from zero to β₃ In Z of having nuclei (A), or a number from zero to 4 in Z having nuclei (B), a number from zero to 5 in Z having nuclei (C), or a number from zero to 6 in Z having nuclei (D);
R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₀ may be ═══O or ═══S; and
-(L)- is a bond or a divalent substituted or unsubstituted chain of from 1 to 10 atoms selected from the group consisting of carbon, nitrogen, sulfur, and oxygen;
Q is a basic group containing one or more basic radicals; and
R₃ is an acidic group containing one or more acid radicals;
or a pharmaceutically-acceptable salt, solvate or pro-drug thereof.

21. The method of claim 19, wherein the spiro compound is represented by the formula: 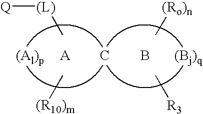
wherein
atoms Aᵢ and Bⱼ are independently selected from carbon, nitrogen, oxygen or sulfur, provided that at least one atom of Aᵢ is carbon, and at least one atom Bⱼ is carbon;
optionally, the rings of the spirobicycle formed by Aᵢ and Bⱼ , respectively, are partly unsaturated;
p and q are Independently numbers from 2 to 6;
m is a number from zero to p;
R₁₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₁₀ may be ═══O or ═══S, If p is 2 or one or two R₁₀ may be ═══O or ═══S, If p is a number from β₃ to 6;
n is the number from zero to q;
R₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₀ may be ═══O or ═══S, If q is 2 or one or two R₀ may be ═══O or ═══S, if q is a number from 3 to 6;
-(L)- is a bond or a divalent substituted or unsubstituted chain of from 1 to 10 atoms selected from the group consisting of carbon, nitrogen, sulfur, and oxygen;
Q is a basic group containing one or more basic radicals; and
R₃ is an acidic group containing one or more acid radicals;
or a pharmaceutically-acceptable salt, solvate or pro-drug thereof.

22. The method of claim 19, wherein the spiro compound is represented by the formula: 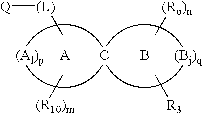
wherein
the spirocycle having (Aᵢ)ₚ, C, and (Bⱼ)_{q} is 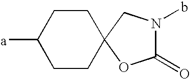
m is a number from zero to 9;
R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;
n is a number from zero to 2;
R₀ ls-the same or different and is a non-Interfering substituent independently selected from alkyl, halosubstituted alkyl, alkenyl, alkynyl, cycloalkyl, aryl, arylalkyl, hydroxy, alkoxy, arylaikoxy, amino, substituted amino, carbamoyl, carboxy, acyl, cyano, halo, nitro, or sulfo;
wherein Q-(L) is attached at a, and 3 is attached at b;
-(L)- is a bond or a substituted or unsubstituted chain selected from the group consisting of CO, CO(C₁-C₆ alkyl), O(C₁-C₆ alkyl), NHCO, and C₁-C₆ alkyl;
Q is a basic group selected from the group consisting of amino, imino, amidino, hydroxyamidino, N-alkylamidine, N,N′-dialkylamidine, N-arylamidine, aminomethyleneamino, aminomethylamino, guanidino, aminoguanidino, alkylamino, dialkylamino, trialkylamino, alkylideneamino, pyrrolyl, Imidazolyl, pyrazolyl, pyridyl, pyrazinyl, pyrimidinyl, indolizinyl, isoindolyl, 3H-indolyl, Indolyl, 1H-indazolyl, purinyl, 4H-quinolizinyl, isoquinolyl, quinolyl, phthalazinyl, naphthyridinyl, quinoxalinyl, quinazolinyl, cinnolinyl, amide, thloamide, benzamidino, pteridinyl, 4aH-carbozolyi, carbozolyl, beta-carbolinyl, phenanthridinyl, acridinyl, phenanthrolinyl, phenazinyl, phenarsazinyl, phenothiazinyl, pyrrolinyl, imidazolidinyl, Imidazolinyl, pyrazolidinyl, pyrazolinyl, piperidyl, piperazinyl, indolinyl, isoindolinyl quinuclidinyl, morpholinyl, any of the foregoing radicals substituted on a benzene ring, optionally substituted with R_{2c}, wherein R_{2c} is hydrogen or halogen and any of the foregoing radicals substituted by amino, imino, amidino, hydroxyamidino, aminomethyleneamino, iminomethylamino, guanidino, alkylamino, dialkylamino, trialkylamino, tetrahydroisoquinoline, dihydrosloindole, alkylideneamino or 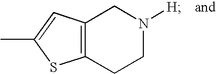
R₃ is an acidic group selected from the group consisting of CO₂ R₅, (C₁-C₆ alkyl)CO₂ R₅, CO(C₁-C₆ alkyl)CO₂ R₅, CONH(C₁-C₆ alkyl)CO₂ R₅, (C₁-C₆ alkyl)CH(NHR₄)CO₂ R₅, CO(C₁-C₆ alkyl)CH(NHR₄)CO₂ R₅, or CONH(C₁-C₆ alkyl)CH(NHR₄)CO₂ R₅, wherein R₄ is SO₂ (C₁-C₆ alkyl), SO₂ aryl, or SO₂ (substituted aryl); and
R₅ is hydrogen, C₁-C₆ alkyl, aryl, or substituted aryl;
or a pharmaceutically-acceptable salt, solvate or pro-drug thereof.

23. The method of claim 19, wherein the spiro compound is represented by the formula: 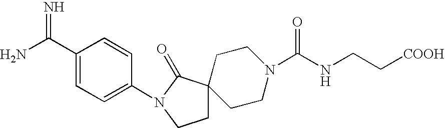
or a pro-drug thereof.

24. The method of claim 23, wherein the pro-drug is represented by the formula:

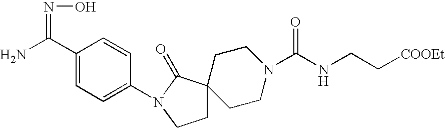

25. A method for destroying a tumor in a subject comprising administering to the subject in need of such therapy a therapeutically effective amount of an activated α_{llb}β₃ receptor antagonist.

26. The method of claim 25, wherein the tumor cell resides in an organ system of the subject.

27. The method of claim 26, wherein the tumor cell resides in a skeletal system of the subject.

28. The method of claim 27, wherein the tumor cell resides in a bone of the subject skeletal system.

29. The method of claim 27, wherein the tumor cell resides in a bone cell of the subject skeletal system.

30. The method of claim 25, wherein the antagonist is a platelet-specific activated α_{IIb}β₃ receptor antagonist.

31. The method of claim 30, wherein the platelet-specific activated α_{IIb}β₃ receptor antagonist is a spiro compound.

32. The method of claim 31, wherein the spiro compound is represented by the formula:
Q-(L)—_{Z}—Z—R₃
wherein
Z is a spirocyclic nucleus selected from the group consisting of Nucleus (A), (B), (C), or (D) represented by the formulas: 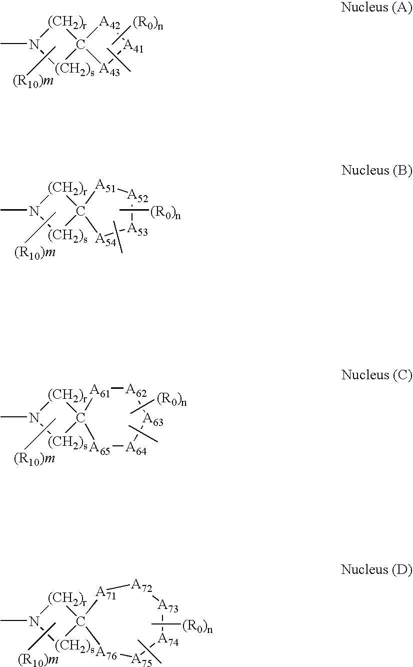
wherein
the group Q-(L)_{Z}- is bound to the nitrogen containing ring of nuclei (A), (B), (C), or (D) and the group R₃ is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆; or
the group R₃ is bound to the nitrogen containing ring and the group Q-(L)_{Z}- is bound to the ring formed by the groups A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆;
r and s are independently a number from zero to 5 with the proviso that not both r or s are 0 and (r+s) is not more than 6, and z is zero or one;
atoms A₄₁, A₄₂, A₄₃, A₅₁, A₅₂, A₅₃, A₅₄, A₆₁, A₆₂, A₆₃, A₆₄, A₆₅, A₇₁, A₇₂, A₇₃, A₇₄, A₇₅, or A₇₆ are independently selected from carbon, nitrogen, oxygen or sulfur, provided that at least one of said atoms is carbon;
the hydrogens of the nitrogen containing part of the spirocycle Z may be substituted by a number of m substituents R₁₀, wherein;
m is a number from zero to (r+s); and
R₁₀ is the same or different and is a non-interfering substituent independently selected from aikyl, halosubstituted aikyl, aikenyl, alkynyl, cycloalkyl, aryl, arylalkyl, hydroxy, alkoxy, arylalkoxy, amino, substituted amino, carbamoyl, carboxy, acyl, cyano, halo, nitro, suifo, ═══O, or ═══S, with the proviso that only one or two RIO may be ═══O or ═══S;
n is a number from zero to 3 In Z of having nuclei (A), or a number from zero to 4 in Z having nuclei (B), a number from zero to 5 in Z having nuclei (C), or a number from zero to 6 in Z having nuclei (D);
R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one or two R₀ may be ═══O or ═══S; and
-(L)- is a bond or a divalent substituted or unsubstituted chain of from 1 to 10 atoms selected from the group consisting of carbon, nitrogen, sulfur, and oxygen;
Q is a basic group containing one or more basic radicals; and
R₃ is an acidic group containing one or more acid radicals;
or a pharmaceutically-acceptable salt, solvate or pro-drug thereof.

33. The method of claim 31, wherein the spiro compound is represented by the formula: 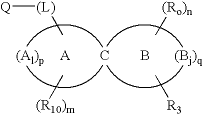
wherein
atoms Aᵢ and Bⱼ are independently selected from carbon, nitrogen, oxygen or sulfur, provided that at least one atom of Aᵢ is carbon, and at least one atom Bⱼ is carbon;
optionally, the rings of the spirobicycle formed by Aᵢ and Bⱼ , respectively, are partly unsaturated;
p and q are independently numbers from 2 to 6;
m is a number from zero to p;
R₁₀ is the same or different and is a non-interfering substituent independently selected from hydrogen, alkyl, halosubstltuted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₁₀ may be ═══O or ═══S, if p is 2 or one or two R₁₀ may be ═══O or ═══S, if p is a number from 3 to 6;
n is the number from zero to q;
R₀ is the same or different and is a non-interfering substituent independently selected from hydrogen, alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, sulfo, ═══O, or ═══S, with the proviso that only one R₀ may be ═══O or ═══S, If q is 2 or one or two R₀ may be ═══O or ═══S, If q is a number from β₃ to 6;
-(L)- is a bond or a divalent substituted or unsubstituted chain of from 1 to 10 atoms selected from the group consisting of carbon, nitrogen, sulfur, and oxygen;
Q is a basic group containing one or more basic radicals; and
R₃ is an acidic group containing one or more acid radicals;
or a pharmaceutically-acceptable salt, solvate or pro-drug thereof.

34. The method of claim 31, wherein the spiro compound is represented by the formula: 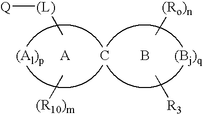
wherein
the spirocycle having (Aᵢ)ₚ, C, and (Bⱼ)_{q} is 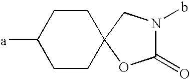
m is a number from zero to 9;
R₁₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;
n is a number from zero to 2;
R₀ is the same or different and is a non-interfering substituent independently selected from alkyl, halosubstituted alkyl, alkenyl, alkynyl, cycloalkyl, aryl, arylalkyl, hydroxy, alkoxy, arylalkoxy, amino, substituted amino, carbamoyl, carboxy, acyl, cyano, halo, nitro, or sulfo;
wherein Q-(L) is attached at a and R₃ is attached at b;
-(L)- is a bond or a substituted or unsubstituted chain selected from the group consisting of CO, CO(C₁-C₆ alkyl), O(C₁-C₆ alkyl), NHCO, and C₁-C₆ alkyl;
Q is a basic group selected from the group consisting of amino, imino, amidino, hydroxyamidino, N-alkylamidine, N,N′-dialkylamidine, N-arylamidine, aminomethyleneamino, aminomethylamino, guanidino, aminoguanidino, alkylamino, dialkylamino, trialkylamino, alkylideneamino, pyrrolyl, imidazolyl, pyrazolyl, pyridyl, pyrazinyl, pyrimidinyl, indolizinyl, isoindolyl, 3H-indolyl, indolyl, 1H-indazolyi, purinyl, 4H-quinolizinyl, isoquinolyl, quinolyl, phthalazinyl, naphthyrldinyl, quinoxalinyl, qulnazolinyl, cinnolinyl, amide, thioamide, benzamidino, pteridinyl, 4aH-carbozolyl, carbozolyl, beta-carbolinyl, phenanthridinyl, acridinyl, phenanthrolinyl, phenazinyl, phenarsazinyl, phenothiazinyl, pyrrollnyl, Imidazolidinyl, imidazolinyl, pyrazolidinyl, pyrazolinyl, piperidyl, piperazinyl, indolinyl, isoindollnyl quinucildinyl, morpholinyl, any of the foregoing radicals substituted on a benzene ring, optionally substituted with R_{2c}, wherein R_{2c} is hydrogen or halogen and any of the foregoing radicals substituted by amino, imino, amidino, hydroxyamidino, aminomethyleneamino, Iminomethylamino, guanidino, alkylamino, dialkylamino, trialkylamino, tetrahydroisoquinoline, dihydrosioindole, alkylideneamino or 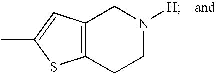
R₃ is an acidic group selected from the group consisting of CO₂ R₅, (C₁-C₆ alkyl)CO₂ R₅, CO(C₁-C₆ alkyl)CO₂ R₅, CONH(C₁-C₆ alkyl)CO₂ R₅, (C₁-C₆ alkyl)CH(NHR₄)CO₂ R₅, CO(C₁-C₆ alkyl)CH(NHR₄)CO₂ R₅, or CONH(C₁-C₆ alkyl)CH(NHR₄)CO₂ R₅, wherein R₄ is SO₂ (C₁-C₆ alkyl), SO₂ aryl, or SO₂ (substituted aryl); and
R₅ is hydrogen, C₁-C₆ alkyl, aryl, or substituted aryl;
or a pharmaceutically-acceptable salt, solvate or pro-drug thereof.

35. The method of claim 31, wherein the spiro compound is represented by the formula: 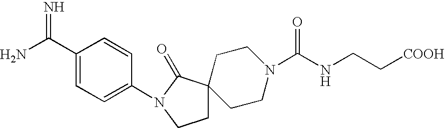
or a pro-drug thereof.

36. The method of claim 35, wherein the pro-drug is represented by the formula:

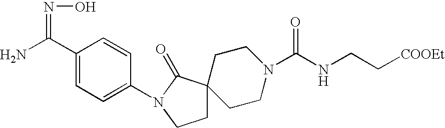

37. A method for treating, preventing or inhibiting tumor cell metastasis to bone in a subject comprising replacing substantially all bone marrow affected by tumor cell metastasis transplant in the subject, wherein said bone marrow is replaced with β₃^{-/-}bone marrow.

38. A method for treating, preventing or reversing tumor metastasis or formation comprising modulating β₃ integrin expression.

39. The method of claim 38, wherein the modulating β₃ integrin expression comprises decreasing the β₃ integrin expression in a mammalian cell.

40. The method of claim 39, wherein decreasing the expression comprises transforming the cell to express a polynucleotide anti-sense to at least a portion of an endogenous polynucleotide encoding β₃ integrin.

41. The method of claim 39, wherein decreasing the expression comprises transfecting the cell with a polynucleotide anti-sense to at least a portion of an endogenous polynucleotide encoding β₃ integrin.

42. The method of claim 39, wherein decreasing the expression comprises transfecting a cell with a siRNA targeting at least a portion of an endogenous polynucleotide encoding β₃ integrin.
